(19) United States
(12) Patent Application Publication
(10) Pub. No.: US 2022/0290184 A1
(43) Pub. Date: Sep. 15, 2022

(54) DONOR PIGS FOR XENOTRANSPLANTATION

(71) Applicant: NZENO LIMITED, Auckland (NZ)

(72) Inventors: Paul Lip Jin TAN, Auckland (NZ); Olga GARKAVENKO, Auckland (NZ); Robert Bartlett ELLIOTT, Auckland (NZ)

(73) Assignee: NZENO LIMITED, Auckland (NZ)

(21) Appl. No.: 17/632,376

(22) PCT Filed: Jul. 31, 2020
(86) PCT No.: PCT/IB2020/057239

§ 371 Date: Feb. 2, 2022

(30) Foreign Application Priority Data
Aug. 6, 2019 (AU) 2019902809

(51) Int. Cl. C12N 15/877; C12N 15/85; C12N 15/90; A01K 67/027

(52) U.S. Cl. CPC C12N 15/8778; C12N 15/8509; C12N 15/907; A01K 67/0276; A01K 2227/108; A01K 2267/025

(57) ABSTRACT

The present invention relates to novel strains of pig that are highly suitable for xenotransplantation. The first novel pig strain lacks functional porcine endogenous retroviruses so is suitable as a donor for tissue and/or cell xenotransplantation into a human recipient. These pigs can also be used as a foundation pig for further manipulation, for example, by gene editing of xenoantigens to produce a second novel strain of pig that is not only free of infectious porcine retroviruses but is also free of the main xenoantigens responsible for hyperacute organ rejection. These pigs can be used for whole organ, tissue and/or cell transplantation into a human recipient. The present invention also relates to methods for selecting pigs that lack infectious porcine endogenous retroviruses, and their use for tissue and/or cell xenotransplantation into humans, and to methods of gene editing of xenoantigens of the selected pigs to further enhance the immunological quality of the donor organs, tissues and/or cells to avoid xenotransplant rejection.

### FIELD OF THE INVENTION

This invention generally relates to novel strains of pig that are highly suitable for xenotransplantation. The first novel pig strain lacks functional porcine endogenous retroviruses so is suitable as a donor for tissue and/or cell xenotransplantation into a human recipient. These pigs can also be used as a foundation pig for further manipulation, for example, by gene editing of xenoantigens to produce a second novel strain of pig that is not only free of infectious porcine retroviruses but is also free of the main xenoantigens responsible for hyperacute organ rejection. These pigs can be used for whole organ, tissue and/or cell transplantation into a human recipient.

The invention also relates to methods for selecting pigs that lack infectious porcine endogenous retroviruses, and their use for tissue and/or cell xenotransplantation into humans, and to methods of gene editing of xenoantigens of the selected pigs to further enhance the immunological quality of the donor organs, tissues and/or cells to avoid xenotransplant rejection.

### BACKGROUND

Xenotransplantation has the potential to solve the worldwide problem of human donor organ and tissue shortages, especially xenotransplantation using organs, tissues and/or cells from pigs.

Pigs are the closest in size and physiology to humans, making their organs, tissue and cells suitable as donors for human transplantation. Pigs are also easy to breed and manage in biosecure facilities. However, a major issue that has so far prevented their wide spread use in xenotransplantation is the fact that pigs are carriers of porcine endogenous retroviruses (PERVs). Most breeds of pigs have PERVs integrated into the genome of all their cells, and these viruses have been shown to be able to infect human cells in vitro. There is a risk therefore that PERVs in pig organs, tissues and cells can be transfected into human recipients, not only infecting the recipients but also potentially spreading infectious diseases to the general population which may cause disease characteristic of retroviruses, such as immunodeficiency and cancer.

There are three PERV subtypes, PERV-A, PERV-B and PERV-C. PERV-A and PERV-B are ubiquitous and can be transmitted to pigs and humans. PERV-C is only able to infect pig cells but can replicate and recombine with PERV-A to produce a highly infectious hybrid PERV-A/C strain (Kimsa et al., 2014). The number of integrated PERVs varies in different pig breeds/strains and ranges from 1 to over 300 copies. Selection of pigs with a very low PERV copy number is one way to produce pigs for xenotransplantation.

Known selection methods of pig breeds for low PERV status has traditionally involved the use of polymerase chain reaction (PCR), using primers that are complementary to a variety of PERV DNA and RNA sequences to identify certain regions of the PERV virus (e.g. gag, pol or env gene sequences). The outcome of PCR testing can be a simple positive or negative result, or can enable the PERV copy number to be calculated. Pigs that have naturally low PERV copy number include Auckland Island (AI) pigs, which have a PERV copy number of between 4 and 40. PCR selection methods have been used to select and breed an AI pig herd with a very low PERV copy number of between 0 and 30 for xenotransplantation (WO 2006/110054). The AI pigs in this herd were also pre-selected for other traits to minimise host rejection such as having blood type O and being free of MHC Class I antigen, as well as being designated pathogen-free (DPF).

These pigs provided one of the first animal populations suitable for xenotransplantation. However, this selection method cannot accurately identify those pigs that have full length PERV capable of replication from those that have inactive PERV, i.e. PERV status or copy number determined by PCR is not associated with virus that is able to replicate. For example, PCR amplification of very small regions of gag, pol or env PERV sequences does not distinguish between full length or partial PERV sequences. A PCR-based test also does not distinguish between an intact gene and a gene that contains nonsense mutation(s) (stop codons). This means that a PERV positive PCR test could be associated with a partial sequence that is not able to replicate, or a full length sequence that contains nonsense mutation(s) (false positives), and a PERV negative result could have missed some of the small env protein gene sequences (false negative).

In addition, a copy number as low as 1 could mean that the organs, tissues and cells transplanted into a human recipient may still have sufficient PERV to replicate and potentially cause disease. Indeed, to avoid this problem, pig cells have had to be protected from the recipient's immune system before implantation, for example by alginate encapsulation or by using an implant device.

Elimination of PERV or at least PERV deactivation to eliminate the ability of PERV to replicate and therefore be infectious, would be a more desirable goal.

Several methodologies to achieve this goal have been tried, including RNA interference (RNAi) vaccines, PERV elimination using zinc finger nucleases or TAL effector nucleases, but have met with limited success.

More recently, the use of CRISPR technology has been suggested as a way of inactivating genome wide PERV via gene editing. Yang et al (2015) succeeded in inactivating 62 PERV sequences in an immortalised pig cell line and Niu et al (2017) have, via CRISPR gene editing in primary fetal pig fibroblast cells and somatic-cell nuclear transfer cloning technology, succeeded in producing piglets with inactive PERVs. This involved inactivating 25 PERV sequences.

The use of CRISPR or any other gene-editing technology to inactivate all PERV genes, in conjunction with somatic-cell nuclear transfer cloning technology, appears to be seen as the best way forward to produce porcine organs for xenotransplantation that are human-compatible. For example, eGenesis (Cambridge, Mass.) are promoting the use of CRISPR Cas-9 technology to produce viable cloned porcine embryos that have been gene edited so that all PERVs are inactivated.

PERV infection is not the only problem. Pigs should also be free of other pathogens that could be transmitted to human recipients, including lymphotrophic herpes virus (PLHV), circovirus (PCV) and cytomegalovirus (PCMV), for example.

Another major hurdle to be overcome is that of immunological incompatibility with the recipient and therefore hyperacute organ rejection. Again, CRISPR or any other gene-editing technology can be used to edit xenoantigen genes (such as alpha 1,3 galactosyltransferase (GGTA1) and cytidine monophosphate-N-acetyl neuramic acid hydroxylase (CMAH) genes, etc) to either remove or deactivate unwanted xenoantigens to reduce immunological adverse reactions. In addition, human genes that moderate the immune system can be added to pigs to help to avoid rejection (Revivicor, Blacksburg, Va.).

However, the combination of the use of gene-editing technology to deactivate/remove all PERV and xenoantigen genes (eGenesis talk about introducing “double digit” gene edits to reduce the likelihood of organ rejection (Weintraub, 2019)) can mean that the resulting cloned animals have dozens of gene edits which may harm the animal and/or have unforeseen genetic consequences.

A better way of producing animals that are PERV-free without having to carry out such multiple gene edits would therefore be preferable and provide animals that do not have the potential problems that large number of gene edits is likely to cause.

It is an object of the present invention to go some way towards overcoming this problem and/or to provide the public with a useful choice.

In this specification where reference has been made to patent specifications, other external documents, or other sources of information, this is generally for the purpose of providing a context for discussing the features of the invention. Unless specifically stated otherwise, reference to such external documents or such sources of information is not to be construed as an admission that such documents or such sources of information, in any jurisdiction, are prior art or form part of the common general knowledge in the art.

### SUMMARY OF THE INVENTION

The present invention provides a use of complete genome sequencing (CGS) as a tool to select PERV-C and PERV-A/C negative pigs, i.e. pigs that have no or inactive, non-replicating PERV C and PERV-A/C, for xenotransplantation of tissues and/or cells into human recipients; and/or as foundation pigs for further manipulation, for example by gene editing xenoantigens, to provide pigs that are not only PERV-A and PERV-A/C negative but are also free of xenoantigens that are responsible for hyperacute organ rejection so that this novel pig strain is suitable as donors for whole organ, tissue and/or cell xenotransplantation into human recipients.

In a first aspect, the present invention provides a method of selecting pigs suitable as donors for xenotransplantation of tissues and/or cells, or as foundation pigs for further manipulation, said method comprising the step:
a) providing a designated pathogen-free (DPF) pig herd having a low PERV copy number of between 4 and 40;
b) testing the PERV status of individual pigs of the herd using complete genome sequencing (CGS);
c) identifying individual pigs that have PERV-C negative and PERV-A/C negative status;
d) selecting said PERV-C negative and PERV-A/C negative pigs as donor pigs for xenotransplantation, or as foundation pigs for further manipulation.

The method preferably further comprises identifying the PERV-A and PERV-B status of the PERV-C negative and PERV-A/C negative pigs of step d) and further selecting pigs that have a very low number of full-length and potentially functional PERV-A and PERV-B sequences of between 1-10, preferably between 1-5, and most preferably between 1-2.

Preferably the pigs are Auckland Island (AI) pigs, a swine breed that has designated pathogen-free (DPF) status and has a surprising low PERV copy number. More preferably, the AI pigs have been selectively bred for very low PERV copy number in a biosecure facility. More preferably, the AI pigs are PERV-null, i.e. any PERV sequences present in their genome are not able to produce infectious PERV particles (Garkavenko et al., 2008).

Other pig breeds/strains that are designated pathogen-free and have similar low PERV copy number and PERV-null status can also be used in the present invention.

In a second aspect the invention provides pigs selected by the method of the first aspect.

It was surprisingly found that full-length and potentially functional PERV-A/B sequences, in the PERV-C and PERV-A/C negative pigs selected by the method of the first aspect, were located on the Y-chromosome only so could be used to breed out functioning PERV. In a third aspect the present invention therefore provides a method of breeding a herd of pigs that have no functional PERV genomic sequences suitable as donors for xenotransplantation, or as foundation pigs for further manipulation, said method comprising the steps:
a) selecting PERV-C and PERV-A/C negative male and female pigs using the CGS selection method of the first aspect;
b) analysing the chromosomal location of any full-length, potentially functional PERV-A and PERV-B gene sequences in the male pigs of step a);
c) selecting male pigs that have full-length, potentially functional PERV-A and/or PERV-B present in the Y-chromosome only;
d) breeding the male pigs of step c) with the female pigs of step a) to produce progeny; and
e) selecting female progeny that will lack the paternal full length and potentially functional PERV-A and/or PERV-B as future breeding stock to produce a herd of pigs that have no functional PERV genomic sequences suitable as donors of tissues and/or cells for xenotransplantation, or as foundation pigs for further manipulation.

The present invention also contemplates selecting the female progeny of step e) as suitable donors for xenotransplantation of tissues and/or cells, or as foundation pigs for further manipulation.

In a fourth aspect the invention provides a pig herd bred by the method of the third aspect.

The foundation pigs selected by the CGS selection method of the first aspect of the invention, or bred by the method of the third aspect of the invention can be further manipulated using gene editing technology to eliminate and/or deactivate one or more xenoantigens selected from GGTA1, CAMH, B4GalNT2, Neu5Gc, ASGR1 and SLA, for example, to minimise the risk of hyperacute rejection of tissues and/or cells, as well as whole organs transplanted into human recipients. In a fifth aspect the invention therefore provides a method of providing donor pigs suitable for xenotransplantation of whole organs, tissues and/or cells into a human recipient comprising the steps:
(a) selecting PERV-C and PERV-A/C negative donor pigs using the CGS method of the first aspect;
(b) establishing a PERV-C and PERV-A/C negative cell line from the pigs of step (a);
(c) gene editing selected cells of step (b) to eliminate or deactivate one or more xenoantigen genes;
(d) optionally gene editing selected isolated cells of step (c) to express one or more human genes selected from A20, CD39, CD46, CD47, CD55, CD59, hemoxygenase-1 (HO-1), HLA-E, HLA-G, thrombomodulin (TM), CTLA4-Ig, and LEA29Y;
(e) establishing a gene-edited cell line from the pig cells of step (c) or step (d);
(f) carrying out somatic cell nuclear transfer from one or more of said gene edited cells of step (e) into an oocyte from a PERV-C and PERV-A/C negative pig in vitro;
(g) culturing to blastocyst stage;
(h) transferring said blastocyst(s) to a female PERV-C and PERV-A/C negative pig and growing to full term; and
(i) providing donor pigs that are PERV-C and PERV-A/C negative and gene edited for one or more xenoantigens.

The one or more xenoantigens genes may be selected from GGTA1, CAMH, B4GalNT2, Neu5Gc, ASGR1 and SLA, or any other xenoantigen that would be useful to reduce immunological reactions, especially the possibility of hyperacute organ rejection, as would be understood by a skilled worker.

In a sixth aspect the invention provides pig cell lines that are PERV-C and PERV-A/C negative and gene edited to inactivate or delete one or more xenoantigens and optionally to express one or more human genes, produced by the method of the fifth aspect. The cell lines can be used to clone donor pigs for xenotransplantation of whole organs, tissues and/or cells into human recipients.

In a seventh aspect the invention provides donor pigs that are DPF, PERV-C and PERV-A/C negative and gene edited to inactivate or delete one or more xenoantigens and optionally to express one or more human genes, produced by the method of the fifth aspect. These donor pigs represent a novel pig strain, designated NZeno-1. These pigs also preferably have a very low number of full-length and potentially functional PERV-A and PERV-B sequences of between 1-10, preferably between 1-5, and most preferably between 1-2.

In an eighth aspect the invention provides tissues and/or cells from the pigs of the second, fourth, and seventh aspects for use in xenotransplantation.

In a ninth aspect the invention provides whole organs from the pigs of the seventh aspect for use in xenotransplantation.

The organs, tissues and/or cells for use in xenotransplantation may be selected from the group consisting of kidney, liver, lung, heart, brain, pancreas, muscle, blood, bone, testes and ovary.

The tissue and/or cells for use in xenotransplantation may be selected from pancreatic islets, hepatocytes, non-parenchymal liver cells, gall bladder epithelial cells, gall bladder endothelial cells, bile duct epithelial cells, bile duct endothelial cells, hepatic vessel epithelial cells, hepatic vessel endothelial cells, sinusoid cells, choroid plexus cells, fibroblasts, Sertoli cells, adrenal chromaffin cells and muscle cells.

In a tenth aspect the invention provides a method of treating a patient suffering from or predisposed to a disease, disorder or condition associated with a deficiency in or absence of organ function, said method comprising transplanting tissue and/or cells of the eighth aspect, or whole organs of the ninth aspect to a patient in need thereof.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed.

These are only examples of what is specifically intended and all possible combinations of numerical values between the lowest value and the highest value enumerated are to be considered to be expressly stated in this application in a similar manner.

As used herein the term ‘(s)’ following a noun means the plural and/or singular form of that noun.

The term ‘comprising’ as used in this specification and claims means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

As used herein the term ‘and/or’ means ‘and’ or ‘or’, or where the context allows both.

The Invention consists in the foregoing and also envisages constructions of which the following gives examples only.

As used herein the terms ‘PERV-C negative’ and ‘PERV-A/C negative’ means no full length proviral genome identified in pigs selected by the method of the invention. This can be distinguished from the terms “PERV-C negative” and “PERV-A/C negative” when assessed by conventional PCR which can only detect the presence of a small part of env region (PERV-C(A/C) positive) or absence of this region (PERV-C(A/C) negative) of PERV-C and recombinant PERV-A/C genomic sequences.

As used herein the term ‘PERV-null’ means the inability to produce infectious PERV particles.

The term “porcine” is used interchangeably with the terms “pig” and “swine” and refers to mammals in the family Suidae. Such mammals include wholly or partially inbred swine, preferably those members of the Auckland Island pig herd described herein.

The term “recipient” as used herein refers to a human suffering from or predisposed to a disease, disorder or condition associated with a deficiency in or absence of organ, tissue or cell function.

The term “gene editing” or “genome editing” is a type of genetic engineering in which DNA is inserted, deleted, modified or replaced in specific targeted sites in the genome of a living organism. Gene editing techniques include the use of engineered nucleases, or ‘molecular scissors’ that create site-specific double stranded breaks at desired locations in the genome. The induced double-strand breaks are repaired through non-homogenous end-joining or homologous recombination, resulting in targeted mutations or gene edits. Engineered nucleases that can be used include zinc finger nucleases, transcription activator-like effector nucleases, engineered meganucleases and the clustered regularly interspaced short palindromic repeats (CRISPR/Cas-9) system.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will now be described by way of example only and with reference to the accompanying drawings in which:

FIG. 1 shows full PERV-C sequence BLAST hits against the Sus scrofa Male1 genome sequence;

FIG. 2 shows a sequence alignment of selected regions of the Sus scrofa Male1 genome with known PERV sequences. Disagreements to the PERV-C reference KC116219 are highlighted in black;

FIG. 3 shows a Maximum Likelihood phylogenetic reconstruction of 8 selected hits of Sus scrofa Male1 (black) with 9 known PERV sequences (red). The tree was rooted with the PERV-B sequence. Numbers at the nodes indicate the ML support values. The scale bar represents the number of expected substitutions per site;

FIG. 4 shows a Maximum Likelihood phylogenetic reconstruction of 18 genomic regions (inconsistently found across these PERV-A, B, C, and A/C references with a coverage threshold of 90%) of Sus scrofa Male1 (black) with 9 known PERV sequences (red). The tree was rooted with the PERV-B sequence. Numbers at the nodes indicate the ML support values. The scale bar represents the number of expected substitutions per site;

FIG. 5 shows a Maximum Likelihood phylogenetic reconstruction of selected gag regions of the Sus scrofa Male1 genome (black) with 9 known PERV sequences (red). The tree was rooted with the PERV-B sequence. Numbers at the nodes indicate the ML support values. The scale bar represents the number of expected substitutions per site;

FIG. 6 shows a Maximum Likelihood phylogenetic reconstruction of selected pol regions of the Sus scrofa Male1 genome (black) with 9 known PERV sequences (red). The tree was rooted with the PERV-B sequence. Numbers at the nodes indicate the ML support values. The scale bar represents the number of expected substitutions per site;

FIG. 7 shows a Maximum Likelihood phylogenetic reconstruction of selected env regions of the Sus scrofa Male1 genome (black) with 9 known PERV sequences (red). The tree was rooted with the PERV-B sequence. Numbers at the nodes indicate the ML support values. The scale bar represents the number of expected substitutions per site;

FIG. 8 shows full PERV-C sequence BLAST hits against the Sus scrofa Male2 genome sequence;

FIG. 9 shows a sequence alignment of selected regions of the Sus scrofa Male2 genome with known PERV sequences. Disagreements to the PERV-C reference KC116219 are highlighted in black;

FIG. 10 shows a Maximum Likelihood phylogenetic reconstruction of 8 selected hits of Sus scrofa Male2 (black) with 9 known PERV sequences (red). The tree was rooted with the PERV-B sequence. Numbers at the nodes indicate the ML support values. The scale bar represents the number of expected substitutions per site;

FIG. 11 shows a Maximum Likelihood phylogenetic reconstruction of 18 genomic regions (inconsistently found across these PERV-A, B, C, and A/C references with a coverage threshold of 90%) of Sus scrofa Male2 (black) with 9 known PERV sequences (red). The tree was rooted with the PERV-B sequence. Numbers at the nodes indicate the ML support values. The scale bar represents the number of expected substitutions per site;

FIG. 12 shows a Maximum Likelihood phylogenetic reconstruction of selected gag regions of the Sus scrofa Male2 genome (black) with 9 known PERV sequences (red). The tree was rooted with the PERV-B sequence. Numbers at the nodes indicate the ML support values. The scale bar represents the number of expected substitutions per site;

FIG. 13 shows a Maximum Likelihood phylogenetic reconstruction of selected pol regions of the Sus scrofa Male2 genome (black) with 9 known PERV sequences (red). The tree was rooted with the PERV-B sequence. Numbers at the nodes indicate the ML support values. The scale bar represents the number of expected substitutions per site;

FIG. 14 shows a Maximum Likelihood phylogenetic reconstruction of selected env regions of the Sus scrofa Male1 genome (black) with 9 known PERV sequences (red). The tree was rooted with the PERV-B sequence. Numbers at the nodes indicate the ML support values. The scale bar represents the number of expected substitutions per site;

FIG. 15 shows the morphology of NZK1 cells cultured on three different substrates;

FIG. 16 shows the proliferation characteristics of NZK1 cells on three different substrates. TC=tissue culture;

FIG. 17 shows the metaphase chromosomes of an NZK1 cell, a representative example of the total number of metaphase chromosomes released from an individual NZK1 cell at passage 3. Individual chromosomes are numbered;

FIG. 18 shows the CRISPR target site in the GGTA1 gene. Cleavage by CRISPR is expected to delete a 152 bp fragment, as indicated, which leads to a functional disruption of the gene;

FIG. 19 shows sequence verification of the AI pig target regions. Shown is the genomic sequence of the relevant target regions of the GGTA1 gene in AI pig cells in comparison to the published pig genome sequence (SEQ ID NOs: 1-6);

FIG. 20 shows sequence comparisons of wild type cells (WT) (SEQ ID NO: 7) and pooled cells that were transfected (TC) with CRISPRs specific for GGTA1. The arrow indicates the predicted CRISPR cleavage site and the bar below the sequence the region of overlapping sequences for the transfected cells;

FIG. 21 shows bioinformatic identification of the different sequences present in amplified target sites of pooled CRISPR-transfected cells. Shown are the results from Tracking of Indels by DEcomposition (TIDE) analysis of cell transfected with two different CRISPRs for the GGTA1 gene. The bar graphs indicate specific percentages of sequences detected with 0 indicating the unchanged wild type sequence and 1 to 5 insertions of 1-5 base pairs and −1 to −10 deletions of 1-10 base pairs, respectively;

FIG. 22 shows the characterisation of GGTA1-KO NZK1 cell clones. A shows PCR amplification products of the GGTA1 target region for individual cell clones (e.g. G53, G61, G12) and non-transfected control cells (WT). B shows a sequence comparison of wild type cells and cell clone G12. The region deleted in cell clone G12 is shown as a dashed black line highlighted in red. CRISPR binding and cleavage sites are indicated by black bars and arrows, respectively (SEQ ID NOs: 8-13);

FIG. 23 shows the CRISPR target site in the CMAH gene. Cleavage by CRISPR is expected to delete an 88 bp fragment, as indicated, which leads to a functional disruption of the gene;

FIG. 24 shows sequence verification of the AI pig target regions. Shown is the genomic sequence of the relevant target regions of the CMAH gene in AI pig cells in comparison to the published pig genome sequence (SEQ ID NOs: 14-19);

FIG. 25 shows sequence comparisons of wild type cells (WT) (SEQ ID NO: 20) and pooled cells that were transfected (TC) with CRISPRs specific for CMAH. The arrow indicates the predicted CRISPR cleavage site and the bar below the sequence the region of overlapping sequences for the transfected cells;

FIG. 26 shows bioinformatic identification of the different sequences present in amplified target sites of pooled CRISPR-transfected cells. Shown are the results from Tracking of Indels by DEcomposition (TIDE) analysis of cell transfected with two different CRISPRs for the CMAH gene. The bar graphs indicate specific percentages of sequences detected with 0 indicating the unchanged wild type sequence and 1 to 5 insertions of 1-5 base pairs and −1 to −10 deletions of 1-10 base pairs, respectively;

FIG. 27 shows the characterisation of CMAH-KO NZK3 cell clones. A shows representative gels with PCR amplification products of the CMAH target region for individual cell clones. Indicated are five cell clones (38, 86, 89, 90 and 104) which have been deemed suitable as CMAH KO clones for the generation of KO pigs. B shows a sequence comparison of wild type cells and cell clone 90. The mutation in clone 90, a 1 bp insertion, at the cleavage site of CRISPR 68rev is highlighted by a black box. CRISPR binding and cleavage sites are indicated by black bars and arrows, respectively (SEQ ID NOs: 21-23);

FIG. 28 shows PCR amplification products of the GGTA1 and CMAH target regions for individual cell clones (e.g. 4.68, 4.10, 4.16)) and control cells (WT);

FIG. 29 shows a sequence analysis of mutations in double KO cell clone 4.16. Shown is a comparison of the target sequences for GGTA1 and CMAH present in wild type cells and double KO candidate cell clone 4.16. Deleted regions are shown as dashed black lines highlighted in red. CRISPR binding and cleavage sites are indicated by black bars and arrows, respectively (SEQ ID NOs: 24-35);

FIG. 30 shows characterisation of GGTA1-KO NZK3 cell clones. A shows PCR amplification products of the GGTA1 target region for individual cell clones with cell clone GA2, GA5 and GA20 showing a single fragment with a putative 152 bp deletion. B shows a sequence comparison of wild type cells and cell clone GA5. The region deleted in cell clone G12 is shown as a dashed black line highlighted in red. CRISPR binding and cleavage sites are indicated by black bars and arrows, respectively (SEQ ID NOs: 36-41); and

FIG. 31 shows the analysis of NZK3 cells (A), GGTA1/CMAH double KO cell clones (B) and human HEK cells for the binding of a fluorescently labelled isolectin that has binding specificity for the αGal epitope.

### DETAILED DESCRIPTION OF A PREFERRED EMBODIMENT

The present invention provides novel strains of pig that are highly suitable for xenotransplantation. The first novel pig strain lacks functional porcine endogenous retroviruses so is suitable as a donor for tissue and/or cell xenotransplantation into a human recipient. These pigs can also be used as a foundation pig for further manipulation, for example, by CRISPR gene editing of xenoantigens to produce a second novel strain of pig that is not only free of functioning porcine retroviruses but is also free of the main xenoantigens responsible for hyperacute organ rejection. These pigs can be used for whole organ, tissue and/or cell transplantation into a human recipient.

More specifically, the present invention is directed to the use of complete genome sequencing (CGS) as a highly sensitive and specific tool to select donor pigs that have no, or inactive, non-replicating PERV genomic sequences, for xenotransplantation of tissues and/or cells into human recipients, or as foundation pigs for further manipulation. CGS can identify all of the PERV sequences in the pig genome, i.e. both partial and full-length sequences, to identify and select for individual pigs that lack functional PERV sequences.

The present CGS selection method overcomes the problems of the prior art methods as the risk of false positives and false negatives associated with PERV status and/or copy number (via PCR) is eliminated, the risk of functional infective PERV sequences being transmitted to the recipient is eliminated, and the pigs have not had to undergo dozens of CRISPR gene edits to inactivate all of the PERV genes so that the potential problems associated with such multiple gene edits on the donor animal is also eliminated. The tissues and/or cells from the pigs selected by the CGS method of the invention can also be transplanted into human recipients directly, without the need for protective barriers against the recipient's immune system, such as alginate capsules, implantation devices etc, but they can also be used with these protective barriers if, after undergoing crossmatch testing, the donor tissue is considered at risk of hyperacute rejection.

Preferably, the present invention uses CGS to select for pigs that are PERV-C and PERV-A/C negative. There are three PERV subtypes, PERV-A, PERV-B and PERV-C which refer to differences in the envelope (env) region of PERV. The viral envelope protein is the major determinant of host range and is essential for infection. PERV-A and PERV-B are ubiquitous and infect humans and pigs while PERV-C only infects pigs, however, PERV-A and PERV-C can recombine to form a highly infectious variant PERV-A/C. Therefore, it is desirable for a donor pig to be PERV-C free or to have only non-replicating PERV-C to avoid any recombination with existing PERV-A, as well as being free of the recombined PERV-A/C.

The present invention therefore provides a method of selecting pigs suitable as donors for xenotransplantation of tissues and/or cells, or as foundation pigs for further manipulation, said method comprising the step:
a) providing a designated pathogen-free (DPF) pig herd having a low PERV copy number of between 4 and 40;
b) testing the PERV status of individual pigs of the herd using complete genome sequencing (CGS);
c) identifying individual pigs that have PERV-C negative and PERV-A/C negative status;
d) selecting said PERV-C negative and PERV-A/C negative pigs as donor pigs for xenotransplantation, or as foundation pigs for further manipulation.

The method further comprises identifying the PERV-A and PERV-B status of the PERV-C and PERV-A/C negative pigs and selecting pigs that have a very low number of full-length potentially functional PERV-A and PERV-B sequences of between 1-10, preferably between 1-5 and most preferably between 1-2.

The donor pigs, prior to selection using the CGS method of the invention, must be designated pathogen free (DPF). In particular, the pigs must be free from infectious microorganisms such as herpesvirus, PLHV, PCMV, hepatitis E virus (HEV), Toxoplasma, eperythrozoon, brucella, listeria, mycobacterium TB, leptospirillium, Haemophilus suis, any virus causing porcine respiratory reproductive syndrome, any virus causing rabies, any virus causing pseudorables, parvovirus, encephalomyocarditis virus, any virus causing swine vesicular disease, porcine polio virus (techen), any virus causing hemagglutinating encephalomyocarditis, swine influenza type A, adenovirus, transmissible gastroenteritis virus and vesicular stomatitis virus. The pigs' pathogen free status should be regularly monitored using techniques known in the art such as immunological assays and PCR technologies.

The donor pigs are also preferably naturally low in PERV copy number or have been pre-selected and bred for low PERV copy number. For example, Auckland Island (AI) pigs are a swine breed that has designated pathogen-free status and has a surprising low PERV copy number. The AI pigs can be further selectively bred for very low PERV copy number in a biosecure facility using PCR/RT-PCR detection of PERV sequence and calculation of PERV copy number, followed by selective breeding of swine with the lowest PERV copy number as described in WO 2006/110054. The AI pigs have also been shown to be PERV-null, i.e. any PERV sequences present in their genome are not able to produce infectious PERV particles (Garkavenko et al., 2008). Other swine breeds/strains that have the same designated pathogen-free status, low PERV copy number, and PERV-null status could also be used in the present invention.

More preferably the donor pigs have also been pre-selected for other traits to minimise host rejection such as having blood type O and being free of MHC Class I antigen prior to selection using the CGS method of the present invention.

The present invention can use any one of the known techniques of complete genome sequencing (CGS) for detecting the PERV status of the donor pig. CGS is a method for determining the genome sequence of an organism. There are several methods known in the art for obtaining genome sequences. One common technique is whole-genome shotgun sequencing (Venter, 1998). This typically involves several steps:
obtaining a large number of short DNA sequences from random locations in the genome,
assembling the short sequences into larger contigs (genome assembly), and
analysing the data produced.

The present invention uses CGS to identify PERV sequences in pigs and analyse those sequences to determine whether the identified PERV sequences are functional, i.e. capable of replication. The identified PERV sequences are compared with reference sequences to determine whether they are full-length and to identify any mutations that may deactivate the PERV. The surrounding genomic context can also be analysed to determine whether the PERV is likely to be transcriptionally silenced.

Pigs selected using the CGS method of the present invention are also provided by the present invention. These pigs can be used as foundation pigs for further manipulation, as described below, or for use as donors of tissue and/or cells for xenotransplantation.

These pigs have advantages over all current pig breeds purportedly suitable as xenotransplant donors, including those AI pigs selected for low PERV copy number described in WO2006/110054. In particular, by using the CGS method of the present invention the selected pigs can be verified to be truly PERV-C and PERV-A/C negative for the first time, i.e. they do not have any functioning genomic PERV-C and recombined PERV-A/C sequences so can be used as donor pigs for tissue and/or cell xenotransplantation without fear of transmitting functioning PERV-C and PERV-A/C sequences to the recipient.

This major benefit of the selection method of the present invention can be seen in examples 1 and 2 of the present invention.

In example 1 of the invention, a male AI pig (Male1) having an apparent PERV copy number of 18 was in fact found to have only two full-length PERVs, one that groups with the PERV-A reference, and one that groups with the PERV-B reference. The remaining 16 PERV sequences, including PERV-C sequences, were found to have multiple nonsense mutations (stop codons) In the gag, pol, and/or env genes. This pig was deemed PERV-C and PERV-A/C negative by conventional PCR methods and this status was verified by the CGS method of the invention. Furthermore, the CGS method of the invention determined that it contained only two potentially functional full length PERV sequences. The accuracy of this determination is a major benefit of the CGS method, as standard PCR-based testing only gives a positive or negative result that is prone to false-positives when PERV genes are present but contain nonsense mutations. When PCR is used to calculate PERV copy number, it can greatly overestimate the true number of functional PERV sequences.

However, as seen in example 2 of the invention, a second male AI pig (Male2) having a PERV-C positive status as determined by conventional PCR-based testing was in fact found to be PERV-C negative, and to have only a single full-length PERV that groups with PERV-A. As in example 1, the remaining PERV sequences, Including PERV-C sequences, were found to have multiple nonsense mutations (stop codons) in the gag, pol, and/or env genes and are therefore inactive. This result was highly surprising. It was not expected that the selection method of the present invention would overturn the PCR based PERV-C positive status of an animal, especially given that the present method affirmed the PCR deemed PERV-C negative status of Male1.

The present invention has further unexpected advantages over the conventional PCR method of determining PERV-C and PERV-A/C status as, even when PCR identifies an animal as PERV-C positive, the present method can further identify whether or not the PERV-C sequences are complete, if they are not, as in the case of the Male2, there can be no PERV-A/C recombinants in the genome.

The selection method of the present invention is therefore highly sensitive and far superior to the previous PCR-based method that had identified donor pig Male2 as being PERV-C positive. Such a donor pig would not have been considered suitable for use in xenotransplantation for fear of transmission of PERV-C and the possibility of the highly infectious PERV-A/C recombinant sequence in the recipient.

The CGS method of the present invention can not only identify and analyse the functional status of all of the PERV gene sequences in the pig genome, but can also identify the PERV gene sequence locations. Surprisingly, in example 2, the single full-length and potentially functional PERV-A sequence found was located on the Y-chromosome. This surprising finding has unforeseen benefits as such animals can be used to breed out functioning PERVs in future offspring by selectively breeding such pigs with female pigs that are PERV-C and PERV-A/C negative (as selected using the CGS method of the invention) and selecting female offspring as suitable donor pigs or for future breeding as their offspring will not have the functional paternal PERV-A. Such selective breeding can eventually produce a PERV-negative herd for xenotransplantation, i.e. a herd of pigs that have no functional PERV genomic sequences.

The present invention therefore provides a method of breeding a PERV negative herd of pigs for xenotransplantation, said method comprising the steps:
a) selecting PERV-C negative and PERV-A/C negative male and female pigs using the CGS selection method of the invention;
b) analysing the chromosomal location of any full-length, potentially functional PERV-A and PERV-B gene sequences in the male pigs of step a);
c) selecting male pigs that have full-length, potentially functional PERV-A and/or PERV-B present in the Y-chromosome only;
d) breeding the male pigs of step c) with the female pigs of step a) to produce progeny; and
e) selecting female progeny that will lack the paternal potentially functional PERV-A and/or PERV-B as future breeding stock to produce a herd of PERV negative pigs suitable as donors of tissues and/or cells for xenotransplantation, or as foundation pigs for further manipulation.

The present invention also contemplates the selection of female progeny of step e) as suitable donors for xenotransplantation of tissues and/or cells, or as foundation pigs for further manipulation.

The present invention also provides a PERV-negative pig herd bred by the breeding method of the invention. This pig herd can be used as foundation pigs for further manipulation or as a source of donor pigs for xenotransplantation of tissues and/or cells into human recipients. Depending on the results of crossmatch testing, the tissues and/or cells may be able to be transplanted directly, without the need for additional immune barriers such as alginate capsules or implantation devices, or such immune barriers may need to be used to avoid hyperacute rejection of the implant.

As mentioned above, the present invention provides foundation pigs that are PERV-C and PERV-A/C negative (i.e. have no full-length or non-functional full-length PERV-C and PERV-A/C genomic sequences) that have been selected by the CGS method of the present invention, and/or bred by the breeding method of the invention. These foundation pigs represent a novel strain of pig having a true PERV-C and PERV-A/C negative genomic status. These foundation pigs can be further manipulated using gene editing technology to eliminate and/or deactivate one or more xenoantigens, thereby providing a novel pig strain (designated as NZeno-1) that is highly suitable for xenotransplantation of whole organs, tissues and cells into human recipients. As would be understood by a skilled worker, gene editing technology can be used to further improve the xenotransplantable status of the foundation pigs, i.e. to make them more compatible to human recipients, i.e. by deactivating and/or eliminating the xenoantigens responsible for hyperacute organ rejection.

For example, suitable cells obtained from the DPF foundation pigs that have been selected for PERV-C and PERV-A/C negative status and preferably having a very low copy number of active PERV-A and PERV-B (1-2) using the CGS selection method of the invention, and/or bred using the breeding method of the invention, can be used to establish a PERV-C and PERV-A/C free cell line. These cells can then be subjected to gene editing technology to knock out or deactivate genes related to xenoantigens known to trigger immune reactions in the recipient that cause hyperacute organ rejection. Hyperacute organ rejection is mediated by natural antibodies to pig antigens, complement fixation to endothelial cells, and the rapid onset of intravascular coagulation. The major target of these antibodies is a specific carbohydrate epitope (αGal) placed on branched sugar chains on cell surfaces by the enzyme alpha 1,3 galactosyl transferase (GGTA1). GGTA1 is not present in humans so the presence of these αGal epitopes triggers this immune response. Removal of GGTA1 in donor pigs will therefore be useful to avoid organ, tissue and/or cell rejection in human recipients.

Other targets to reduce xenoreactive antibody binding to the xenografted organ triggering hyper acute organ rejection include β1,4 N-acetylgalactosaminyl transferase (B4GalNT2), CMP-Neu5AC Hydroxylase (CMAH), swine leucocyte antigen (SLA), N-acetylneuraminic acid (Neu5Gc), ASGR1, as well as other target immunoreactive genes as would be understood by a skilled worker.

Other gene edits may be made to avoid organ, tissue and/or cell rejection in human recipients, such as expression of human A20, CD39, CD46, CD47, CD55, CD59, hemoxygenase-1 (HO-1), HLA-E, HLA-G, thrombomodulin (TM), CTLA4-Ig, and/or LEA29Y, etc as would be understood be a skilled worker.

A cell line is established from these gene edited cells, and the gene edited cells are subjected to somatic-cell nuclear transfer to clone donor pigs that are ideally suited to xenotransplantation as, not only are they PERV-C and PERV-A/C negative, but they are also hypoimmunogenic as the gene editing will help to prevent adverse immunological reactions when organs, tissues and/or cells of the cloned donor pigs are implanted into a human donor and avoid hyperacute organ rejection.

Suitable cells include fibroblasts, kidney-derived primary cells, or hepatocytes, for example. Other suitable donor cells should have at least the following properties: (1) exhibit a correct and stable karyotype, which could be simply determined by counting the chromosome numbers in metaphase spreads, (2) show good proliferation, (3) have a long lifespan, and (4) should be transferable. Furthermore, an in vitro SCNT experiment gives information about developmental competence until blastocyst stage, as would be understood by a skilled worker. The cells are then gene edited, using CRISPR Cas-9 technology, and subsequent somatic-cell nuclear transfer is carried out, using known methods (Kurome et al., 2015).

The present invention therefore provides a method of providing a novel strain of donor pigs suitable for xenotransplantation of whole organs, tissues and/or cells into a human recipient comprising the steps:
a) selecting foundation pigs that are PERV-C negative and PERV-A/C negative using the CGS selection method of the invention;
b) establishing a PERV-C and PERV-A/C negative pig cell line from the pigs of step a);
c) gene editing selected isolated cells of said pigs of step b) to eliminate or deactivate one or more xenoantigen genes;
d) optionally gene editing selected isolated cells of step c) to express one or more human genes selected from A20, CD39, CD46, CD47, CD55, CD59, hemoxygenase-1 (HO-1), HLA-E, HLA-G, thrombomodulin (TM), CTLA4-Ig, and LEA29Y;
e) establishing a gene-edited cell line from the cells of step c) or d);
f) carrying out somatic cell nuclear transfer from one or more of the gene edited cells of step e) into an oocyte of a pig that is PERV-C negative and PERV-A/C negative;
g) culturing to blastocyst stage;
h) transferring blastocyst to a female PERV-C and PERV-A/C negative pig and growing to full term; and
i) providing a novel strain of donor pigs that are PERV-C and PERV-A/C negative and gene edited for one or more xenoantigens, and optionally gene edited to express one or more human genes.

The one or more xenoantigen genes may be selected from GGTA1, CAMH, B4GalNT2, Neu5Gc, ASGR1 and SLA, and other xenoantigen genes that would be beneficial to deactivate, to improve acceptance by the recipient's immune system as would be understood by a skilled worker.

The present invention also provides pig cell lines produced by step e) of the above method. Examples of pig cell lines of the present invention are set out in examples 4-8 below. Specifically, cell lines from DPF AI pigs selected by the CSG method of the invention (i.e. PERV-C and PERV-A/C negative) were produced that have been gene edited using CRISPR Cas-9 to disrupt GGTA1 (examples 4 and 7); that have been gene edited using CRISPR Cas-9 to disrupt CMAH (example 5); and that have been gene edited using CRISP Cas-9 to disrupt both GGTA1 and CMAH (examples 6 and 8). The cell lines of the present invention can be used to clone donor pigs using known somatic cell transfer techniques (example 9). The cloned donor pigs can then be used for xenotransplantation of whole organs, tissues and/or cells into human recipients. The cloned donor pigs represent a novel pig strain (NZeno-1) having PERV-C and PERV-A/C negative genomic status, a very low number (1-2) of full-length potentially functional genomic PERV-A and PERV-B sequences, having gene edits for one or more xenoantigens, and optionally having one or more gene edits to express human immune-compatible genes. The NZeno-1 donor pigs are also DPF and PERV-null making them particularly suitable for whole organ, tissue and/or cell xenotransplantation as they have been selected and manipulated to minimise the risk of hyperacute organ rejection.

The NZeno-1 novel donor pig strain of the present invention, produced by the combined method of CGS selection for PERV-C and PERV-A/C negative status and low (1-2) PERV A and B, being DPF and PERV-null, and having one or more of GGTA1, CAMH, B4GalT2, Neu5Gc, ASGR1 and/or SLA eliminated or deactivated via gene editing technology, are far superior to any currently available pigs in terms of their suitability as donors for xenotransplantation.

As discussed above, the current thinking in terms of producing pigs as a source of organs, tissues and/or cells for xenotransplantation is to use CRISPR Cas-9 technology to gene edit not only the xenoantigens but also all of the genome wide PERV genes. This would, however require dozens of gene edits being carried out on donor pig cells (usually fibroblasts) prior to somatic cell nuclear transfer and cloning of the resulting embryos. For example, Yang et al (2015) succeeded in inactivating 62 PERV sequences in an immortalised pig cell line and Niu et al (2017) succeeded in inactivating 25 PERV genes to produce piglets with inactive PERVs. Additional gene edits on top of the PERV gene edits would be required to inactivate the xenoantigens (eGenesis talk about introducing “double digit” gene edits to reduce the likelihood of organ rejection (Weintraub, 2019)).

As is now know, CRISPR Cas-9 technology can result in unwanted DNA deletions and rearrangements near its target site on the genome. CRISPR Cas-9 gene editing relies on the Cas-9 enzyme to cut DNA at a particular target site. The cell then attempts to reseal this break using DNA repair mechanisms. The repair mechanisms do not always work perfectly and sometimes segments of DNA will be deleted or rearranged or other unwanted changes can occur. Clearly the more gene edits that are carried out on a single cell, the more the chances of these unwanted edits occurring increases.

The present method produces PERV-C and PERV-A/C negative pigs, without having to carry out gene editing and only contemplates using gene editing to eliminate or deactivate a minimum number of xenoantigen genes thereby avoiding having to produce cloned animals having dozens of gene edits which may harm the animal and/or have unforeseen genetic consequences.

The present invention therefore provides PERV-C and PERV-A/C negative pigs selected by the CGS selection method of the invention, preferably also having a very low copy number of active PERV-A and PERV-B of 1-2, as donor pigs for xenotransplantation of tissues and/or cells or as foundation pigs for further manipulation.

The foundation pigs of the invention may be further manipulated or modified by undergoing gene editing to eliminate or inactivate one or more xenoantigens selected from GGTA1, CAMH, B4GalNT2, Neu5Gc, ASGR1 and SLA, and optionally to express one or more human genes selected from A20, CD39, CD46, CD47, CD55, CD59, hemoxygenase-1 (HO-1), HLA-E, HLA-G, thrombomodulin (TM), CTLA4-Ig, and LEA29Y, to produce a novel NZeno-1 strain.

The CGS selection method of the present invention used to identify PERV-C and PERV-A/C negative status can be carried out before or after gene editing of said one or more xenoantigens.

Usually, gene editing of one or more xenoantigens will be carried out on suitable cells of pigs that have been pre-selected as PERV-C and PERV-A/C negative using the CGS method of the present invention, whereby the gene edited cells are then subjected to somatic cell nuclear transfer to produce cloned pigs that are PERV-C and PERV-A/C negative and have had one or more xenoantigens eliminated/deactivated (novel NZeno-1 strain).

As the pigs used in the present invention are also designated pathogen-free, are PERV-null and may also have been pre-selected for blood group O and to be free of immunogenic antigens present at the cell surface such as MHC Class I antigen, the organs, tissues and/or cells of the present invention are particularly suitable for xenotransplantation. It is anticipated that the organs, tissues and/or cells of the novel NZeno-1 strain of donor pigs of the present invention will be able to be transplanted into most human recipients in need of such transplants as the majority of the immune-mediated responses responsible for hyperacute organ rejection, and which are usually required to be tested for using complex crossmatching, will have been minimised by elimination or deactivation of the major xenoantigens responsible for such immune rejection and by insertion of genes that express human coagulation factors and/or inhibitors of inflammation.

The present invention also provides tissues and/or cells isolated from one or more pigs selected by the CGS method of the present invention. The present invention also provides whole organs, tissues and/or cells isolated from the novel NZeno-1 strain of donor pigs that, in addition to being selected by the CGS selection method of the invention, have also undergone CRISPR Cas-9 gene editing of one or more xenoantigens.

Preferably, the organs, tissues and/or cells are isolated from one or more pigs, wherein said one or more pigs are size matched for the recipient. For example, organs and tissues for transplantation into adult human recipients are preferably isolated from adult pigs, and organs and tissues for transplantation into children are isolated from young/juvenile pigs as would be understood by a skilled worker. Cells for transplantation into adult or children human recipients are preferably isolated from young/juvenile pigs.

The organs, tissues and/or cells may be selected from the group consisting of kidney, liver, lung, heart, brain, pancreas, muscle, blood, bone, testes and ovary.

Preferably, the organs for whole organ xenotransplantation are selected from kidney, liver, lung and heart.

Preferably, the tissue and/or cells for xenotransplantation are selected from pancreatic islets, hepatocytes, non-parenchymal liver cells, gall bladder epithelial cells, gall bladder endothelial cells, bile duct epithelial cells, bile duct endothelial cells, hepatic vessel epithelial cells, hepatic vessel endothelial cells, sinusoid cells, choroid plexus cells, fibroblasts, Sertoli cells, adrenal chromaffin cells and muscle cells.

The invention also provides a method of treating a patient suffering from or predisposed to a disease, disorder or condition associated with a deficiency in or absence of organ function, said method comprising transplanting an organ, tissue and/or cells of the invention to a patient in need thereof.

Such transplantation will preferably be able to restore or augment cell, tissue or organ function in a human recipient whilst minimising the risk of transmission of xenozoonotic infectious agents, including PERV as well as minimising the risk of hyperacute organ rejection.

Preferred embodiments of the invention have been described by way of example only and modifications may be made thereto without departing from the scope of the invention.

### EXAMPLES

### 1. Example 1

This example describes methods for the analysis of PERV content of Sus scrofa by complete genome sequencing.

### 1.1 Methods

Sus scrofa Male1 is a male AI pig that is designated pathogen-free (DPF) and has been identified as PERV-C negative by PCR-based test.

Complete genome sequencing of Sus scrofa Male1 was performed under contract by Macrogen Inc. The Binary Alignment Map (BAM) file (showing filtered DNA sequence reads that are mapped to a reference genome with duplicate reads removed) and Variant Call Format (VCF) file (showing DNA sequence variants that are identified by analysing the information taken from aligned reads) were obtained from Macrogen Inc. and used for further analysis. The files were used to create a consensus sequence using BCFtools and the regions with no coverage from the consensus were replaced with the character “N”. The full PERV-C sequence of KC116219 (8678 bp; NCBI reference sequence) was BLASTed against the Sus scrofa Male1 genome using Geneious.

### 1.2 Results

1.2.1 Genome Sequencing and Identification of PERV Sequences

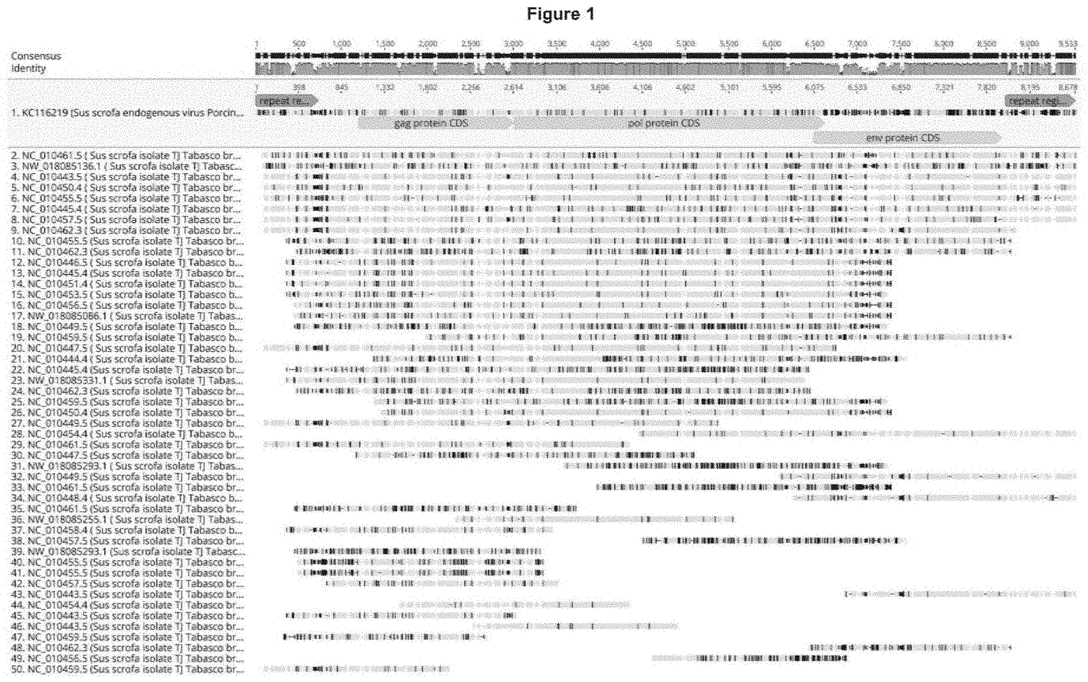

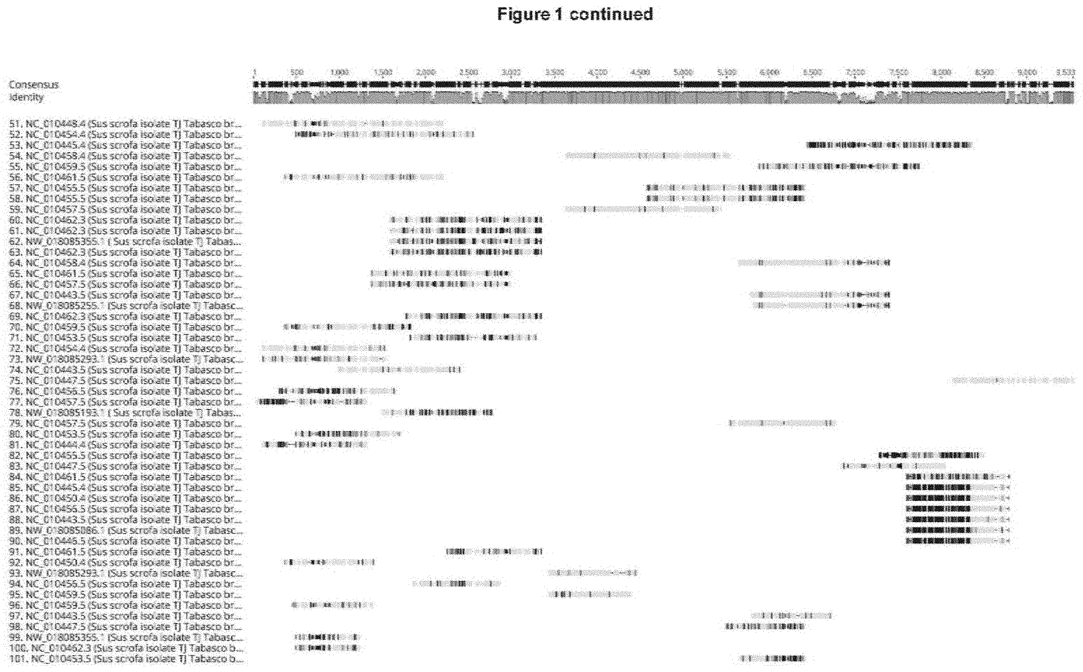

A total of 27 genomic regions covering more than 50% of the query (KC116219) were detected (FIG. 1), and only 8 covered more than 90%. These were chosen as candidates of potentially functional PERVs for further analysis (Table 1).

TABLE 1
Genomic regions of Sus scrofa Male1 that cover
more than 90% of the PERV-C sequence (KC116219).

| | | Hit | Hit | Hit | Query | Query | Query |
|---|---|---|---|---|---|---|---|
| Sequence | Chromosome | length | start | end | coverage | start | end |
| NC_010461.5 | X | 8740 | 73780807 | 73772068 | 99.16% | 74 | 8678 |
| NW_018085136.1 | unplaced | 8755 | 572572 | 563818 | 99.16% | 74 | 8678 |
| | scaffold | | | | | | |
| NC_010443.5 | 1 | 8722 | 262273484 | 262282205 | 99.03% | 85 | 8678 |
| NC_010450.4 | 8 | 8722 | 51596579 | 51605300 | 99.03% | 85 | 8678 |
| NC_010455.5 | 13 | 8711 | 142113106 | 142104396 | 99.03% | 85 | 8678 |
| NC_010445.4 | 3 | 8722 | 10677380 | 10668659 | 99.03% | 85 | 8678 |
| NC_010457.5 | 15 | 8699 | 66922015 | 66913317 | 99.03% | 85 | 8678 |
| NC_010462.3 | Y | 8154 | 25246277 | 25254430 | 92.54% | 85 | 8115 |

1.2.2 Phylogenetic Analysis

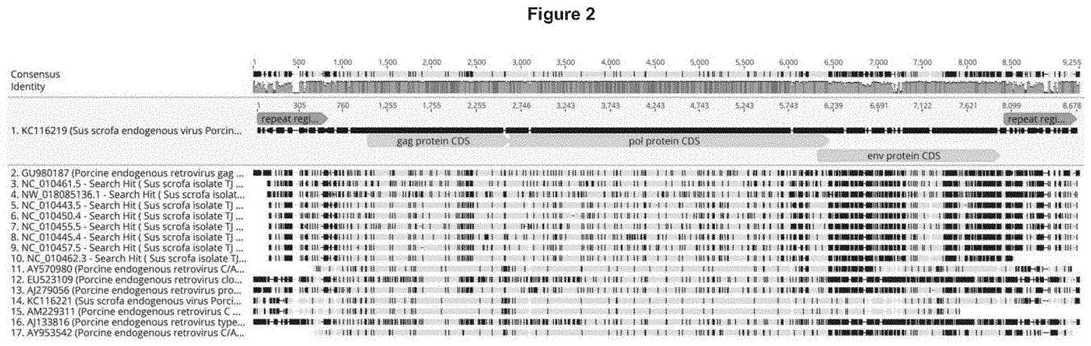

MAFFT was used to make an alignment (FIG. 2) of the 8 hits with 9 existing PERV reference sequences for variants A, B, and C (variant C: KC116219, KC116221, AM229311; recombinant variant A/C: AY570980, AY953542; variant A: AJ279056, GU980187; variant B: EU523109, AJ133816) (Tang et al., 2016; Xiang et al., 2013).

In addition, equivalent analyses were performed using PERV-A, B, C, and A/C references as well. When the coverage threshold was raised to 60%, the same 25 genomic regions were found consistently in the BLAST results. However, when the coverage threshold was raised to 90%, 18 genomic regions were inconsistently found across these PERV-A, B, C, and A/C references.

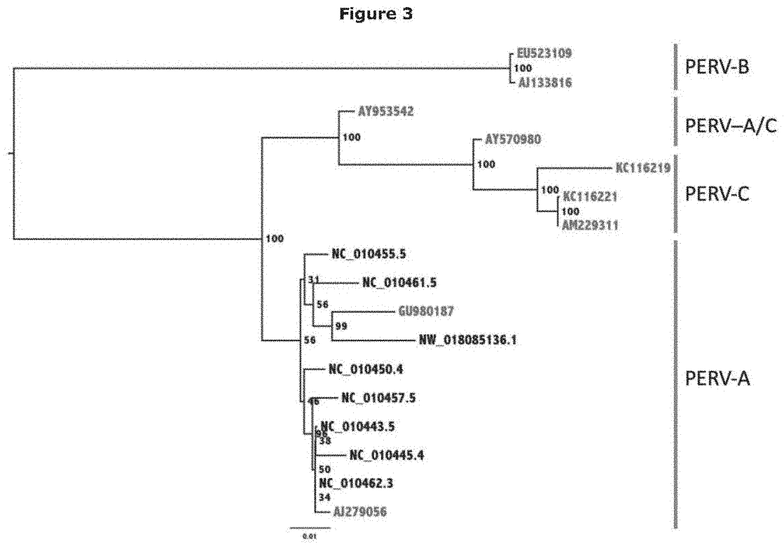

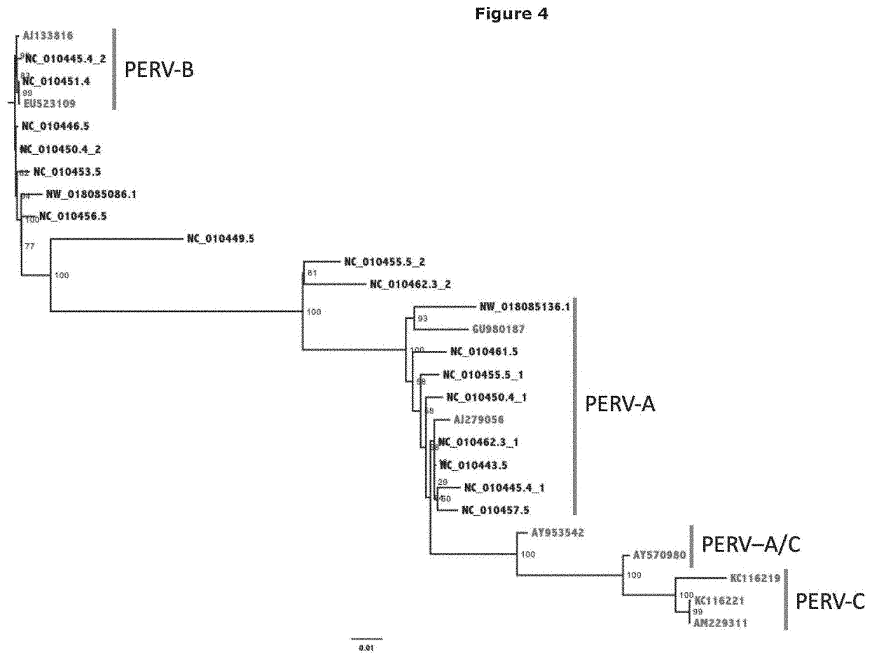

A phylogeny of the 8 hits with greater than 90% coverage of the PERV-C sequence (KC116219) was constructed along with the 9 existing PERV reference sequences to identify the closest PERV variant of each genomic region by IQ-TREE. The phylogenetic results indicated that the 8 hits group with 2 PERV-A reference sequences, which means that all these 8 regions of the Male1 genome show greater similarity to PERV-A (FIG. 3). Moreover, we also conducted a phylogenetic analysis of the 18 genomic regions that were inconsistently found across the PERV-A, B, C, and A/C references with a coverage threshold of 90%. The results revealed that 8 genomic regions (the same regions identified with greater than 90% coverage of the PERV-C sequence) again group with the 2 PERV-A reference sequences, and no genomic regions were grouped with PERV-C and PERV-C(FIG. 4).

1.2.3 PERV Gene Analysis

Protein coding regions of these 18 hits were identified by ORF Finder implemented by the NCBI website with standard genetic codes. The results showed that 16 sequences contained features associated with of loss of function, and only two sequences (NC_010462.3 on chromosome and NC_010445.4_2 on chromosome 3) were found to include full lengths of the 3 retroviral genes (gag, pol, and env) (Table 2).

TABLE 2
Protein coding regions of selected 18 hits of Sus scrofa Male1. Two reference
sequences of PERV-C (KC116219) and PERV-A (GU980187) are included.

| | Whole | gag gene | | | pol gene | | | env gene | | |
|---|---|---|---|---|---|---|---|---|---|---|
| | length | Size | Start | Stop | Size | Start | Stop | Size | Start | Stop |
| | (BP) | (bp) | codon | codon | (bp) | codon | codon | (bp) | codon | codon |
| KC116219 | 8678 | 1566 | ATG | TAG | 3585 | GGA | TAA | 1917 | ATG | TAG |
| (Reference, | | | | | | | | | | |
| PERV-C) | 8939 | 1575 | ATG | TAG | 2931 | ATG | TAG | 1983 | ATG | TAG |
| GU980187 | | | | | | | | | | |
| (Reference, | | | | | | | | | | |
| PERV-A) | | | | | | | | | | |
| NC_010461.5 | 8740 | several stop codons | | | several stop codons | | | several stop codons | | |
| | | found | | | found | | | found | | |
| NW_018085136.1 | 8755 | several stop codons | | | several stop codons | | | several stop codons | | |
| | | found | | | found | | | found | | |
| NC_010443.5 | 8722 | 1575 | ATG | TAG | several stop codons | | | 1614 | ATG | TAG |
| | | | | | found | | | | | |
| NC_010450.4 | 8722 | 1575 | ATG | TAG | several stop codons | | | 1965 | ATG | TAA |
| | | | | | found | | | | | |
| NC_010455.5 | 8711 | several stop codons | | | 3000 | TTG | TAG | 1965 | ATG | TAA |
| | | found | | | | | | | | |
| NC_010445.4 | 8722 | 1575 | ATG | TAG | several stop codons | | | several stop codons | | |
| | | | | | found | | | found | | |
| NC_010457.5 | 8699 | several stop codons | | | 3411 | ATG | TAA | several stop codons | | |
| | | found | | | | | | found | | |
| NC_010462.3 | 8154 | 1575 | ATG | TAG | 3435 | ATG | TAA | 1962 | ATG | TAG |
| NC_010446.5 | 8759 | several stop codons | | | 3411 | ATG | TAG | several stop codons | | |
| | | found | | | | | | found | | |
| NC_010445.4_2 | 8760 | 1575 | ATG | TAG | 2925 | TTG | TGA | 1974 | ATG | TAG |

| | | | | | | | | |
|---|---|---|---|---|---|---|---|---|
| NC_010451.4 | 8757 | 1575 | ATG | TAG | 3411 | ATG | TAG | several stop codons |
| | | | | | | | | found |

| NC_010453.5 | 8758 | several stop codons | 3444 | ATG | TAG | several stop codons | | |
|---|---|---|---|---|---|---|---|---|
| | | found | | | | found | | |
| NW_018085086.1 | 8721 | several stop codons | several stop codons | | | several stop codons | | |
| | | found | found | | | found | | |
| NC_010456.5 | 8679 | several stop codons | several stop codons | | | 1974 | ATG | TAG |
| | | found | found | | | | | |
| NC_010449.5 | 8826 | several stop codons | several stop codons | | | several stop codons | | |
| | | found | found | | | found | | |
| NC_010450.4_2 | 8741 | May have, but many | several stop codons | | | 1974 | ATG | TAG |
| | | N found inside | found | | | | | |
| NC_010455.5.2 | 7707 | several stop codons | 3444 | ATG | TAA | 1983 | ATG | TAG |
| | | found | | | | | | |

| | | | | | | |
|---|---|---|---|---|---|---|
| NC_010462.3_2 | 7698 | several stop codons | 3114 | ATG | TGA | several stop codons |
| | | found | | | | found |

Further analyses were focused on searching the three PERV genes independently. Each gene of the reference sequence KC116219 (gag 1566 bp, env 1917 bp, and pol 3585 bp) was employed to run a search BLAST against the Sus scrofa Male1 genome:

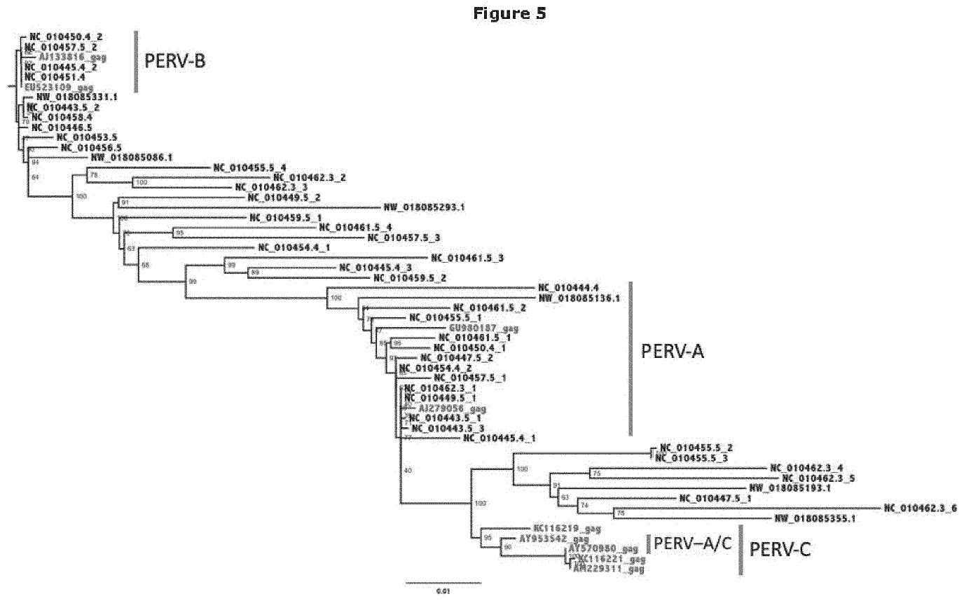

gag: 32 genomic regions were found with query coverage >=90%, but only 15 regions included the full-length gag gene. A total of 45 sequences with >=70% query coverage were selected for phylogeny analysis (FIG. 5).

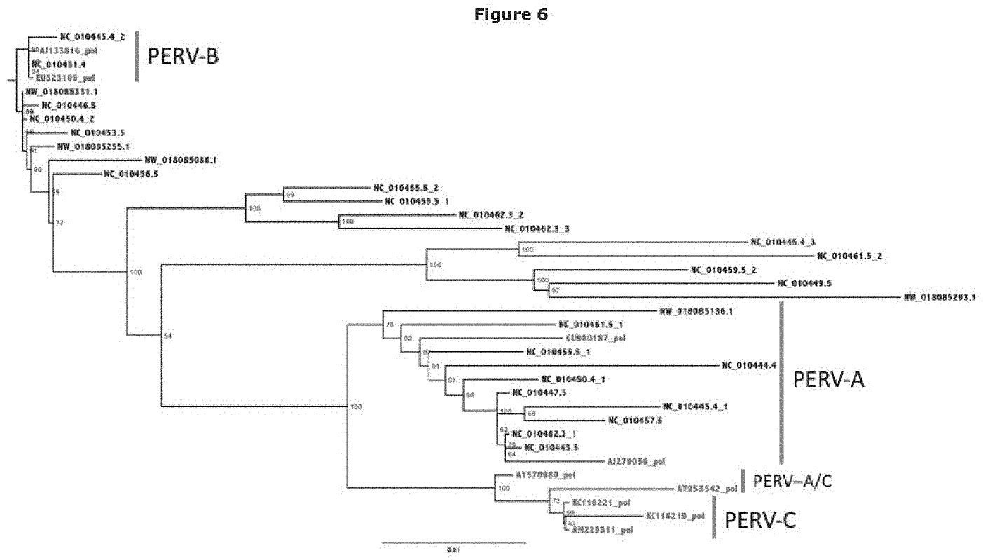

pol: 25 genomic regions were found with query coverage >=90%, but only 11 regions included the full-length pol gene. A total of 28 sequences with >=70% query coverage was selected for phylogeny analysis (FIG. 6).

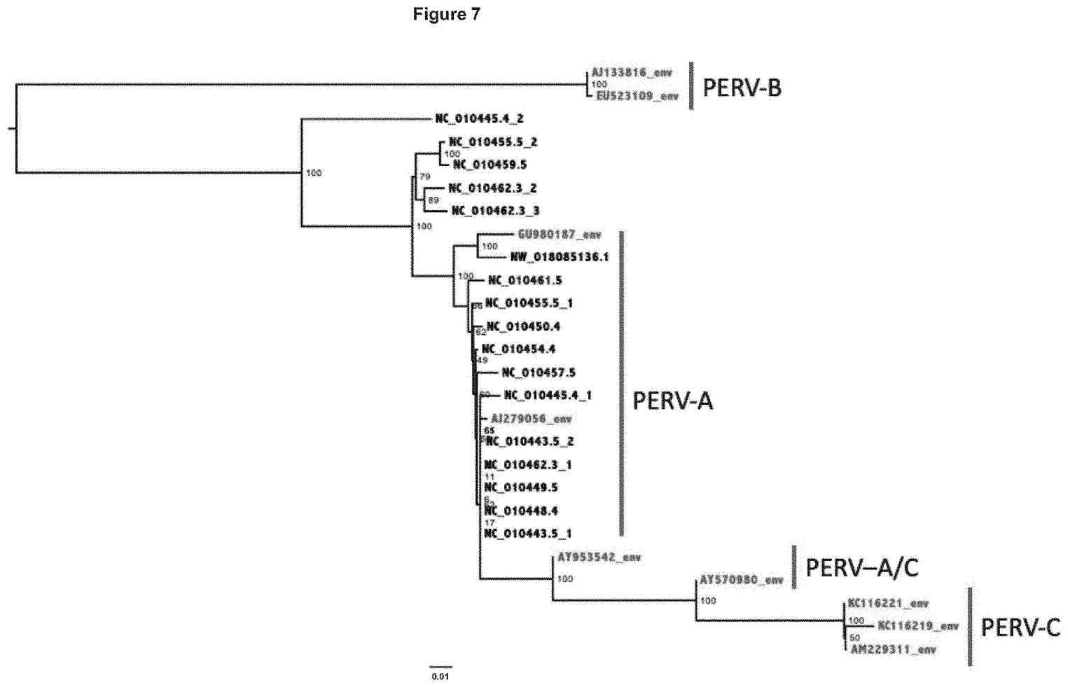

env: 15 genomic regions were found with query cover >=90%, but only 4 regions included the full length env gene. A total of 17 sequences with >=70% query coverage was selected for phylogeny analysis (FIG. 7).

PERV long terminal repeats (LTR) analysis was also carried out and confirmed the results above (results not shown).

### 1.3 Conclusion

For the Sus scrofa Male1 genome, 25 genomic regions covering at least 60% of the PERV sequence were found consistently in the BLAST results of four searches performed with the PERV-A, B, C, and A/C references (FIG. 1). The 8 hits with greater than 90% coverage of PERV-C showed greater similarity to the PERV-A sequences than the PERV-B, C, or A/C sequences (FIG. 3). The 18 genomic regions that were inconsistently found across the PERV-A, B, C, and A/C references using a coverage threshold of 90% did not group with the PERV-A/C or PERV-C sequences (FIG. 4). Only two sequences (NC_010462.3 on chromosome Y and NC_010445.4_2 on chromosome 3) were found to include full lengths of the 3 retroviral genes (gag, pol, and env) (Table 2). Moreover, the sequence of NC_010462.3 was grouped with PERV-A, and the sequence of NC_010445.4_2 was closest to PERV-B. Furthermore, after building the phylogeny of three PERV genes (gag, pol, and env) independently, the results showed that no genomic regions were found to group with the reference genes from PERV-A/C or PERV-C(FIGS. 5-7).

These analyses showed that there is no evidence of functional PERV-A/C or PERV-C sequences in the Sus scrofa Male1 genome. This agrees with the PCR-based test that showed Male1 to be PERV-C negative.

From PERV analysis, without functional gene analysis (equivalent to PERV copy number analysis using RT-PCT) It appears that there are 18 PERVs in the genome of this animal. Such an animal would not have been deemed suitable for xenotransplantation as the risk of PERV-C and/or PERV-A/C infection in the human recipient would have been too high.

However, due to this new selection method, pigs that were previously thought to be unavailable for xenotransplantation due to the presence of active PERV, can now be used as donor pigs for xenotransplantation if the PERV copy number corresponds to non-functional sequences as identified by CGS, or can be used as foundation pigs for further manipulation by gene editing technology as described below.

### 2. Example 2

This example describes an additional instance of the analysis of PERV content of Sus scrofa by complete genome sequencing.

### 2.1 Methods

Sus scrofa Male2 is an AI male pig that is designated pathogen-free (DPF) and has been identified as PERV-C positive by PCR-based test. The complete genome sequencing of Sus scrofa Male2 was performed as described in Example 1 section 1.2.

### 2.2 Results

2.2.1 Genome Sequencing and Identification of PERV Sequences

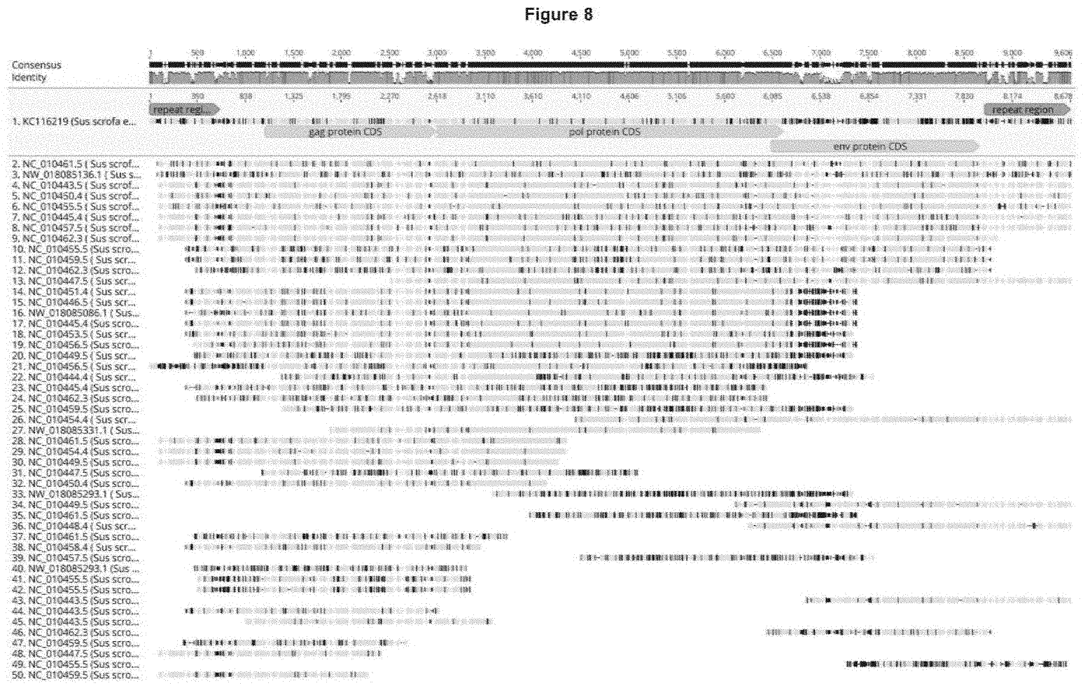

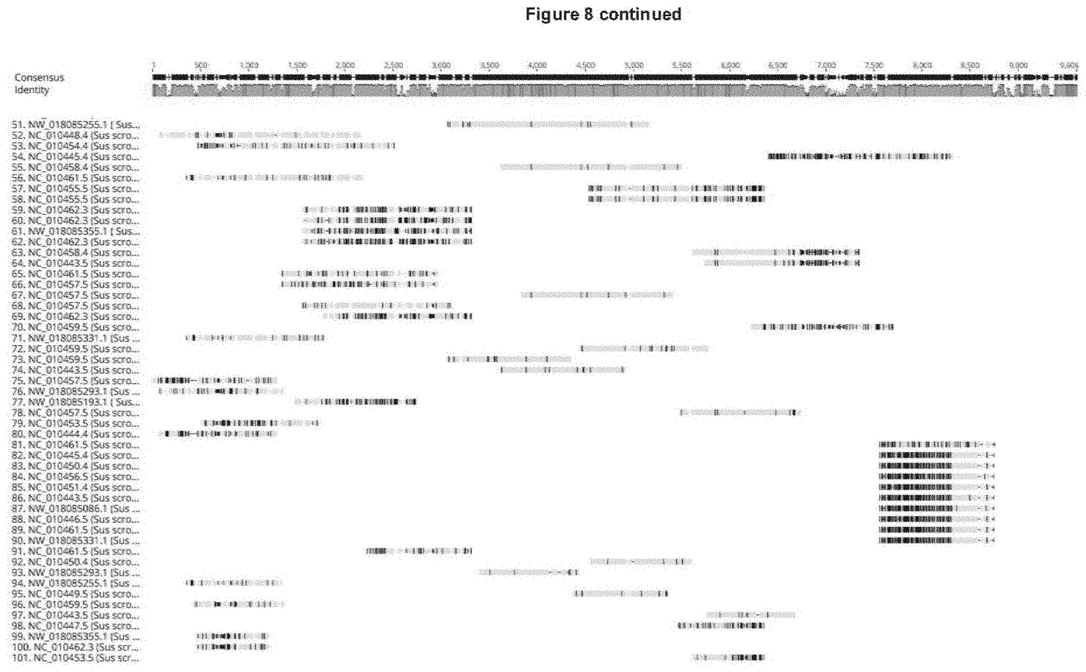

A total of 25 genomic regions covering more than 50% of the query (KC116219) were detected (FIG. 8), and only 8 covered more than 90%. These were chosen as candidates of potentially functional PERVs for further analysis (Table 3).

TABLE 3
Genomic regions of Sus scrofa Male2 that cover
more than 90% of the PERV-C sequence (KC116219).

| | | Hit | Hit | Hit | Query | Query | Query |
|---|---|---|---|---|---|---|---|
| Sequence | Chromosome | length | start | end | coverage | start | end |
| NC_010461.5 | X | 8740 | 73779160 | 73770421 | 99.16% | 74 | 8678 |
| NW_018085136.1 | Unplaced | 8755 | 572583 | 563829 | 99.16% | 74 | 8678 |
| | scaffold | | | | | | |
| NC_010443.5 | 1 | 8722 | 262259539 | 262268260 | 99.03% | 85 | 8678 |
| NC_010450.4 | 8 | 8721 | 51599368 | 51608088 | 99.03% | 85 | 8678 |
| NC_010455.5 | 13 | 8711 | 142091853 | 142083143 | 99.03% | 85 | 8678 |
| NC_010445.4 | 3 | 8722 | 10677954 | 10669233 | 99.03% | 85 | 8678 |
| NC_010457.5 | 15 | 8699 | 66913201 | 66904503 | 99.03% | 85 | 8678 |
| NC_010462.3 | Y | 8156 | 25245370 | 25253525 | 92.57% | 85 | 8117 |

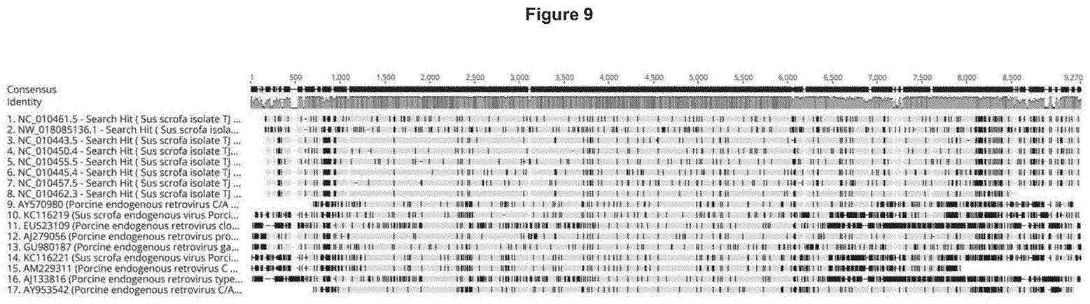

MAFFT was employed to make an alignment (FIG. 9) of the 8 hits with 9 existing PERV reference sequences for variants A, B, and C (variant C: KC116219, KC116221, AM229311; recombinant variant A/C: AY570980, AY953542; variant A: AJ279056, GU980187; variant B: EU523109, AJ133816) (Tang et al., 2016; Xiang et al., 2013).

In addition, equivalent analyses were performed using PERV-A, B, C, and A/C references as well. When the coverage threshold was raised to 60%, the same 25 genomic regions were found consistently in the BLAST results, with the exception that the sequence NC_010447.5 could not be detected using the PERV-B reference sequence. However, when the coverage threshold was raised to 90%, 18 genomic regions were inconsistently found across these PERV-A, B, C, and A/C references.

2.2.2 Phylogenetic Analysis

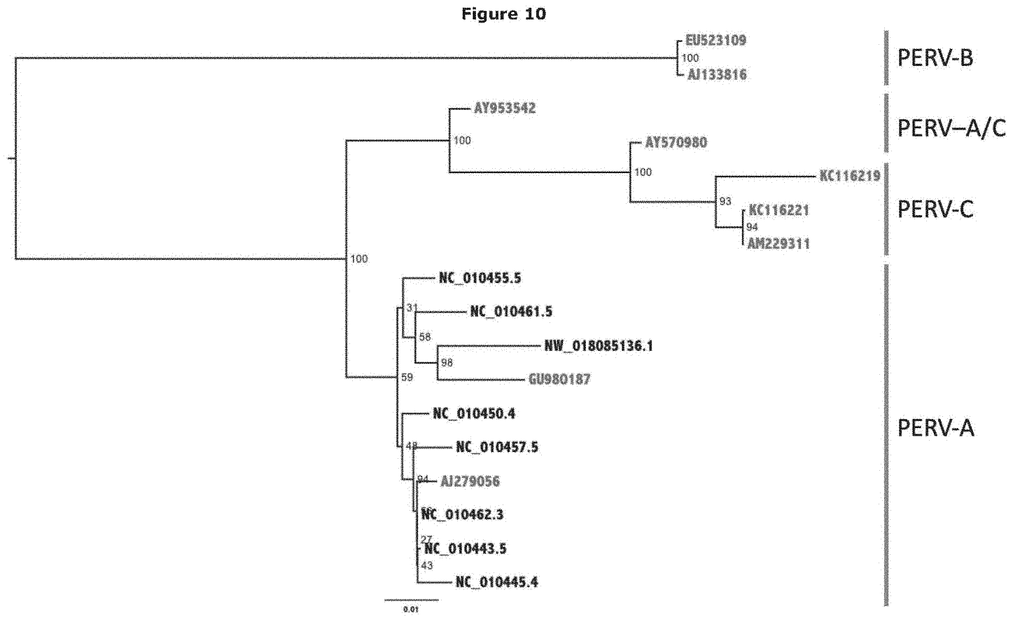

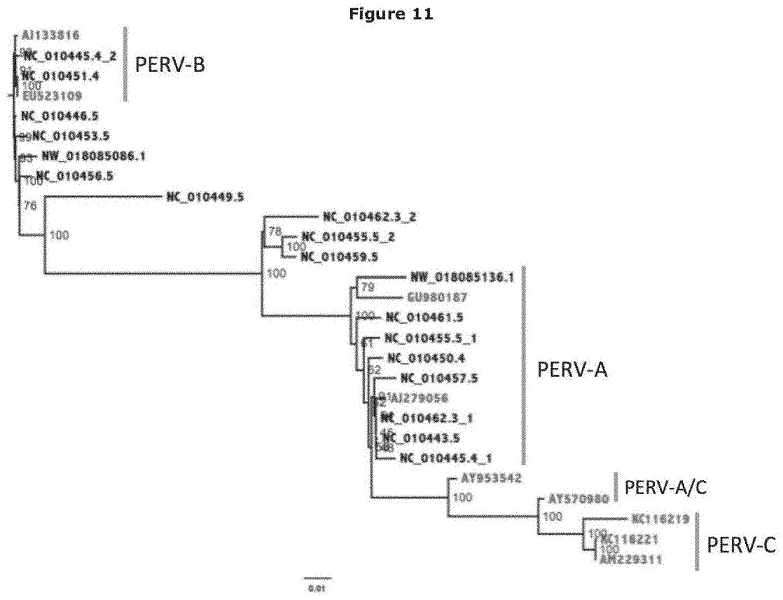

A phylogeny of the 8 hits with greater than 90% coverage of the PERV-C sequence (KC116219) was constructed along with the 9 existing PERV reference sequences to identify the closest PERV variant of each genomic region by IQ-TREE. The phylogenetic results indicated that the 8 hits group with 2 PERV-A reference sequences, which means that all these 8 regions of Male2 genome show greater similarity to PERV-A (FIG. 10). Moreover, we also conduct a phylogenetic analysis of the 18 genomic regions that were inconsistently found across the PERV-A, B, C, and A/C references with a coverage threshold of 90%. The results revealed that 8 genomic regions (the same regions identified with greater than 90% coverage of the PERV-C) group with the 2 PERV-A reference sequences, and no genomic regions were grouped with PERV-A/C and PERV-C(FIG. 11).

2.2.3 PERV Gene Analysis

Protein coding regions of these 18 hits were identified by ORF Finder implemented by the NCBI website with standard genetic codes. The results showed that 17 sequences contained features associated with loss of function, and only the sequence NC_010462.3 on chromosome Y was found to include full lengths of the 3 retroviral genes (gag, pol, and env) (Table 4).

TABLE 4
Protein coding regions of selected 18 hits of Sus scrofa Male2. Two reference
sequences of PERV-C (KC116219) and PERV-A (GU980187) are shown.

| | Whole | gag gene | | | pol gene | | | env gene | | |
|---|---|---|---|---|---|---|---|---|---|---|
| | length | Size | Start | Stop | Size | Start | Stop | Size | Start | Stop |
| | (BP) | (bp) | codon | codon | (bp) | codon | codon | (bp) | codon | codon |
| KC116219 | 8678 | 1566 | ATG | TAG | 3585 | GGA | TAA | 1917 | ATG | TAG |
| (Reference, | | | | | | | | | | |
| PERV-C) | | | | | | | | | | |
| GU980187 | 8939 | 1575 | ATG | TAG | 2931 | ATG | TAG | 1983 | ATG | TAG |
| (Reference, | | | | | | | | | | |
| PERV-A) | | | | | | | | | | |
| NC_010461.5 | 8740 | several stop codons | | | several stop codons | | | several stop codons | | |
| | | found | | | found | | | found | | |
| NW_018085136.1 | 8755 | several stop codons | | | several stop codons | | | several stop codons | | |
| | | found | | | found | | | found | | |
| NC_010443.5 | 8722 | 1575 | ATG | TAG | several stop codons | | | 1614 | ATG | TAG |
| | | | | | found | | | | | |
| NC_010450.4 | 8721 | 1545 | TTG | TAG | several stop codons | | | 1965 | ATG | TAA |
| | | | | | found | | | | | |
| NC_010455.5 | 8711 | several stop codons | | | 3000 | TTG | TAG | 1965 | ATG | TAA |
| | | found | | | | | | | | |
| NC_010445.4 | 8722 | 1575 | ATG | TAG | several stop codons | | | several stop codons | | |
| | | | | | found | | | found | | |
| NC_010457.5 | 8699 | several stop codons | | | 3411 | ATG | TAA | several stop codons | | |
| | | found | | | | | | found | | |
| NC_010462.3 | 8156 | 1575 | ATG | TAG | 3435 | ATG | TAA | 1965 | ATG | TAG |

| NC_010451.4 | 8757 | 1575 | ATG | TAG | 3441 | ATG | TAG | several stop codons |
|---|---|---|---|---|---|---|---|---|
| | | | | | | | | found |
| NC_010446.5 | 8759 | several stop codons | | | 3441 | ATG | TAG | several stop codons |
| | | found | | | | | | found |
| NC_010445.4_2 | 8760 | 1575 | ATG | TAG | 2925 | TTG | TGA | May have, but many |
| | | | | | | | | N found inside |

| NC_010453.5 | 8756 | several stop codons | 3054 | ATG | TAG | several stop codons | | |
|---|---|---|---|---|---|---|---|---|
| | | found | | | | found | | |
| NW_018085086.1 | 8719 | several stop codons | several stop codons | | | several stop codons | | |
| | | found | found | | | found | | |
| NC_010456.5 | 8680 | several stop codons | 3054 | ATG | TAG | 1974 | ATG | TAG |
| | | found | | | | | | |
| NC_010449.5 | 8825 | several stop codons | several stop codons | | | several stop codons | | |
| | | found | found | | | found | | |
| NC_010455.5_2 | 7707 | several stop codons | 3444 | ATG | TAA | 1983 | ATG | TAG |
| | | found | | | | | | |

| | | | | | | |
|---|---|---|---|---|---|---|
| NC_010462.3_2 | 7698 | several stop codons | 3114 | ATG | TGA | several stop codons |
| | | found | | | | found |
| NC_010459.5 | 7695 | several stop codons | 3117 | TTG | TAA | several stop codons |
| | | found | | | | found |

Further analyses were focused on searching the three PERV genes independently. Each gene of the reference sequence KC116219 (gag 1566 bp, env 1917 bp, and pol 3585 bp) was employed to run a search BLAST against the Sus scrofa Male2 genome:

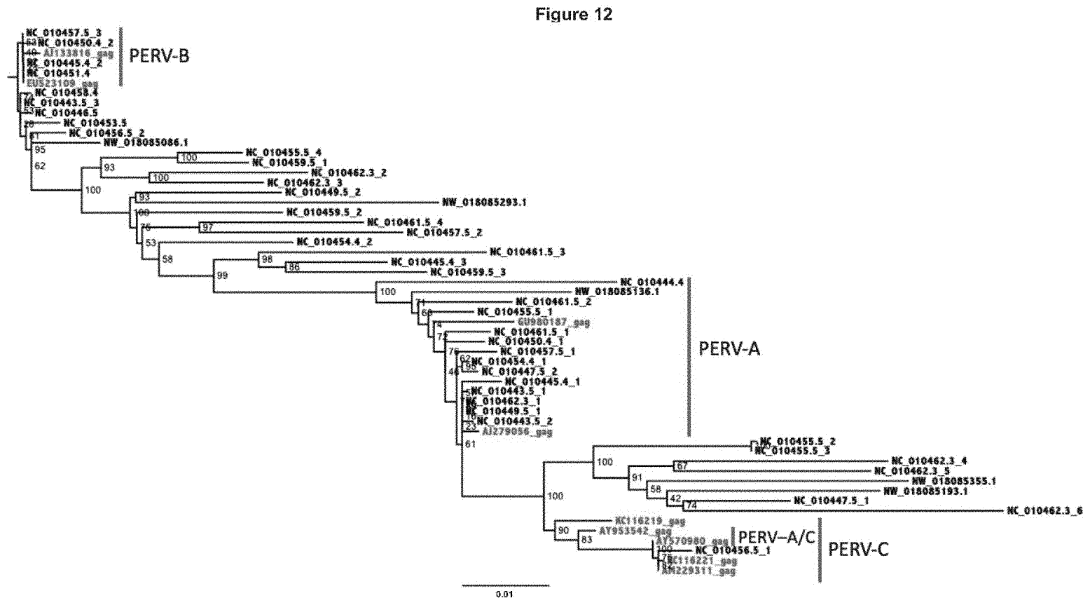

gag: 34 genomic regions were found with query coverage >=90%, but only 14 regions included the full-length gag gene. A total of 46 sequences with query coverage >=70% were selected for phylogeny analysis (FIG. 12).

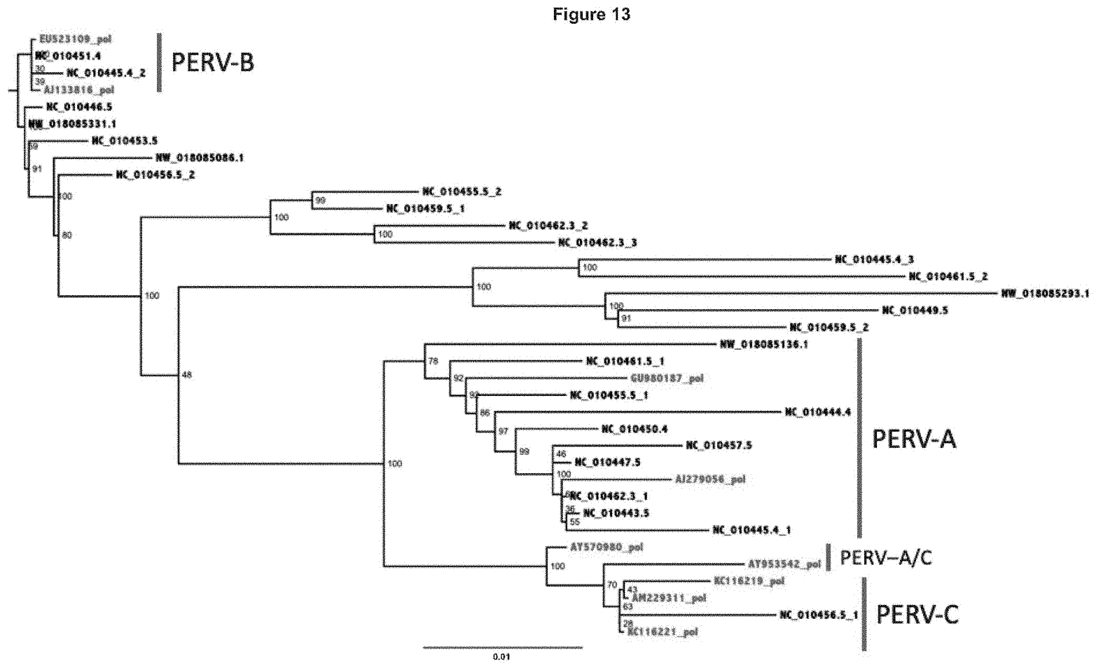

pol: 25 genomic regions were found with query coverage >=90%, but only 11 regions included the full-length pol gene. A total of 27 sequences with query coverage >=70% were selected for phylogeny analysis (FIG. 13).

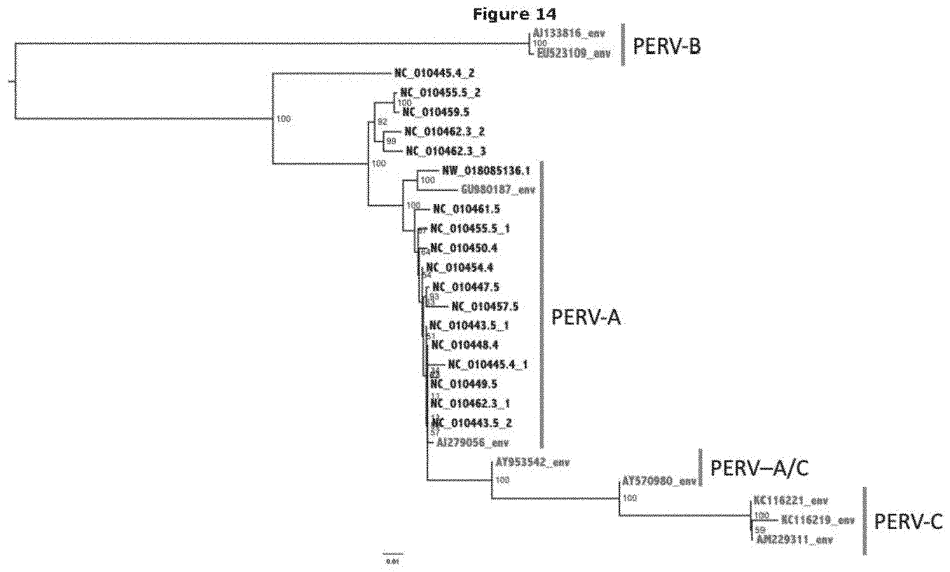

env: 16 genomic regions were found with query coverage >=90%, but only 4 regions included the full length env gene. A total of 18 sequences with query coverage >=70% were selected for phylogeny analysis (FIG. 14). PERV long terminal repeats (LTR) analysis was also carried out and confirmed the results above (results not shown).

### 2.3 Conclusion

For the Sus scrofa Male2 genome, 24 genomic regions covering at least 60% of the PERV sequence were found consistently in the BLAST results of four searches performed with the PERV-A, B, C, and A/C references, with the sole exception that the NC_010447.5 sequence could not be detected by the PERV-B reference. Only one hit (NC_010456.5; chromosome 14, position 62,583,591-62,590,005) showed high similarity to the PERV-C gag gene, but this only partially matched the PERV reference sequence of KC116219 with 73.91% coverage (FIG. 8). The 8 hits with greater than 90% coverage of PERV-C showed greater similarity to the PERV-A sequences than the PERV-B, C, or A/C sequences (FIG. 10). The 18 genomic regions that were inconsistently found across these PERV-A, B, C, and A/C references using a coverage threshold of 90% did not group with the PERV-A/C or PERV-C sequences (FIG. 11). Only one sequence (NC_010462.3 on chromosome Y) was detected to include full lengths of the 3 retroviral genes (gag, pol, and env) (Table 4). Moreover, the sequence of NC_010462.3 was grouped with PERV-A. Furthermore, after building the phylogeny of three PERV genes (gag, pol, and env) Independently, the results showed that no genomic regions were found to group with the reference genes from PERV-A/C (FIGS. 5-7).

Surprisingly, these analyses show that Sus scrofa Male2 is in fact PERV-C negative, despite the previous positive PCR-based test. This new selection method shows that there is no evidence of functional PERV-A/C or PERV-C sequences in the Sus scrofa Male2 genome, indicating that this individual is a candidate as a donor pig for tissues and/or cells for xenotransplantation into a human recipient, or as a foundation pig for further manipulation by gene editing technology as described below.

### 3. Example 3

This example describes the creation and characterisation of the NZK1 cell line derived from the kidneys of Auckland Island (AI) pigs.

### 3.1 Methods

3.1.1 isolation of Kidney Derived AI Pig Cells

Kidney-derived primary AI pig cells were isolated from tissue samples from two individual animals. One was a male newborn piglet (#18/PA007) that accidentally died shortly after birth on 6 Feb. 2018, with the resulting cell line denoted NZK1.

Kidney cells were isolated based on methods from Richter et al (2012). Briefly, 1 cm³ cubes of tissue from the cortex and medulla of the kidney were first washed and then centrifuged after mincing. Afterwards, the tissue was resuspended in 15 ml Hank's Buffered Salt Solution with 0.1% (w/v) collagenase (type I or II; Invitrogen) and incubated at 37° C. while stirring for 1 to 1.5 h. The supernatant, containing kidney cells, was filtered through a 100-μm mesh and washed with Dulbecco's Modified Eagle's Medium (DMEM)+GlutaMAX (Gibco) and centrifuged again (5-10 min, 180×g). The resulting cell pellet was seeded onto tissue culture plates or flasks. The remaining undigested tissue pieces were further treated with 0.25% trypsin/0.02% EDTA for 15 min at 37° C. to harvest additional cells. Kidney cells were cultured in DMEM+GlutaMAX with non-essential amino acids (Gibco), 0.1 mM 2-mercaptoethanol (Gibco) and 10% fetal calf serum (FCS). For the first few days media included 1% (v/v) Penicillin G/Streptomycin and Amphotericin B.

The population doubling time of primary AI kidney cells over continuous passages was determined by culturing NZK1 cells on various substrates; Including directly on tissue culture plastic, as well as coatings of 0.1% gelatin (Sigma) or 0.01% collagen (Sigma).

Mitotic cell spreads were prepared to determine chromosome numbers of NZK1 cells.

An additional cell line was established from the kidney of a female AI piglet using similar methods, denoted NZK3.

### 3.2 Results

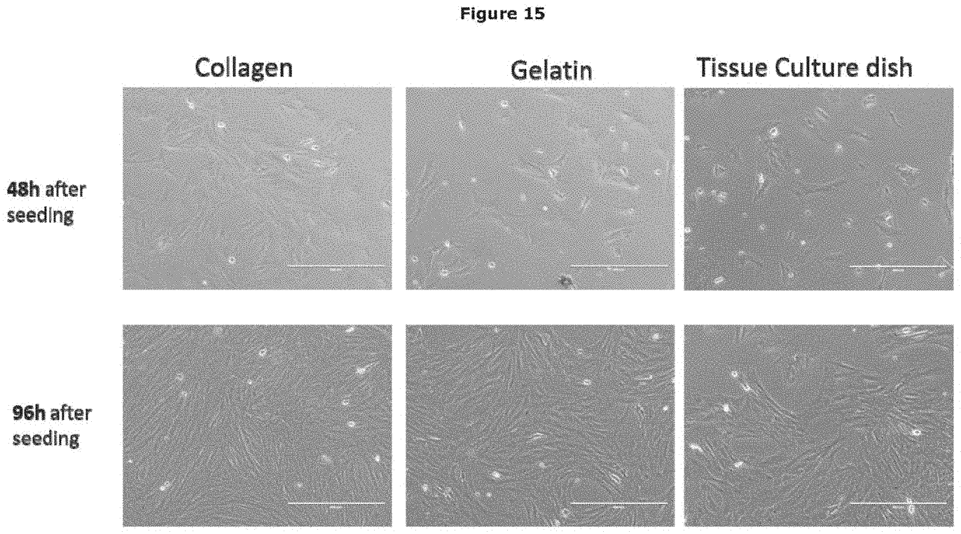

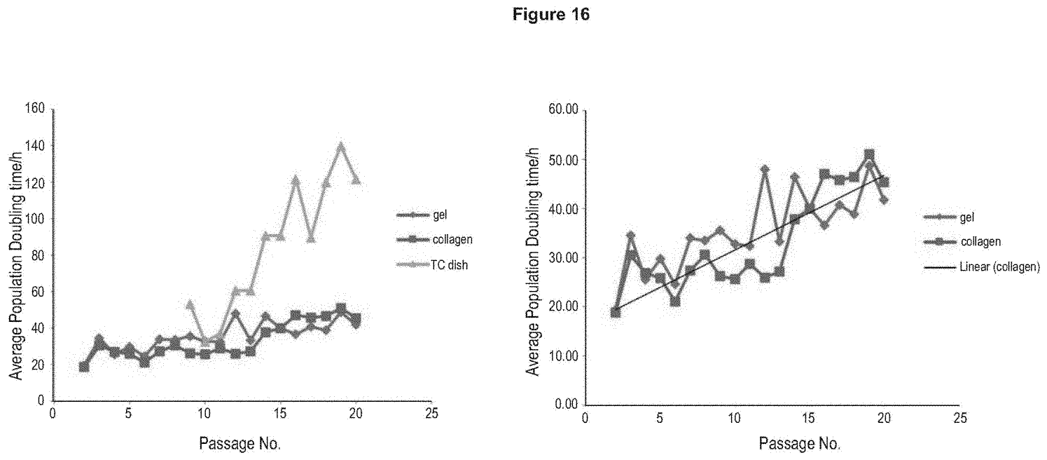

After seeding 1×10⁵ NZK1 cells/3 cm dish over repeated passages, the population doubling time (PDT) of NZK1 cells on tissue culture plastic steadily increased compared to cells grown on either gelatin or collagen coated dishes (FIGS. 15 and 16). The average PDT for the first five passages was 25.6 h. Although PDT on gelatin and collagen dishes had increased to 44 h between passages 15-20 of culture, it was 2.6-fold greater with cells grown on tissue culture plastic.

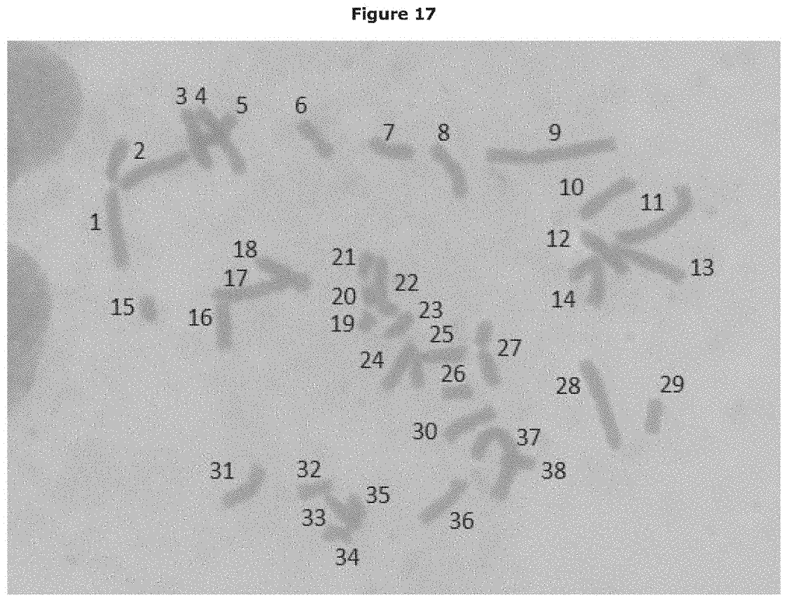

NZK1 cells at passage 3 displayed a mostly diploid chromosome number (2N=38, FIG. 17).

### 4. Example 4

This example describes the creation of a line of Auckland Island (AI) pig cells with a functional disruption to the GGTA1 gene, and so lack the αGal xenoantigen. This cell line was created from the NZK1 cells described in Example 3.

### 4.1 Methods

4.1.1 Nucleic Add Constructs

Two guide RNAs (gRNAs) were designed using the CRISPOR design tool (Haeussler et al., 2016) with target specificity for GGTA1 exon 8 (65fw-CTCTCGTAGGTGAACTCGTC (SEQ ID NO: 42) and 208rev-GATGCGCATGAAGACCATCG (SEQ ID NO: 43), respectively) beginning at position 261,513,520 and 261,513,686, respectively, of Ensemble gene ENSSSCG00000005518.

DNA oligo nucleotides for both strands of the gRNA were synthesised with short adapters, annealed and cloned into the expression vector pX330 according to published protocols (Ran et al., 2013).

4.1.2 Cell Culture and Transfection

isolated kidney-derived primary AI pig cells as described in Example 3 were cultured in DMEM+GlutaMAX with non-essential amino acids, 0.1 mM 2-mercaptoethanol and 10% FCS on culture surfaces that were coated with 0.1% gelatine (Sigma-Aldrich) at 37° C. for 1 h prior to use. CRISPR plasmids were delivered into cells using the Neon transfection system according to the manufacturer's instruction (Invitrogen). Briefly, AI pig cells were harvested by trypsin digestion, washed with calcium and magnesium free Dulbecco's phosphate buffered saline (DPBS) (Sigma-Aldrich) and centrifuged. Approximately 2×10⁶ cells were suspended in 120 μl of resuspension buffer (Invitrogen) containing a total of 5 μg of plasmid DNA when using a single and two CRISPR plasmids and 10 μg with three and four CRISPR plasmids. AI pig cells were electroporated with a 100 μl tip using the Neon program A3 (1500 V pulse voltage, 20 ms pulse width, 1 pulse). After transfection cells were seeded into gelatine coated plates in media containing Pen Strep (Gibco) and cultured with 5% CO₂ at 37° C.

4.1.3 CRISPR Activity Assay

Kidney-derived primary AI pig cells transfected with expression plasmids for GGTA1-specific CRISPRs (65fw, 208rev) were harvested 48 h after transfection. Approximately 1×10⁴ transfected cells were incubated in 20 μl lysis buffer (0.2 μg/ml proteinase K (Qiagen) in PCR buffer (10 mM Tris-HCl, 50 nM KCl, 1.5 mM MgCl₂, pH 8.3; Roche) for 30 minutes at 50° C. followed by 10 minutes incubation at 95° C. to inactivate the reaction. The crude lysate (2 μl) was mixed with 18 μl Kapa 2G Fast Hotstart Ready mix (Kapa Biosystems) to PCR amplify the GGTA1 target locus with GGTA1 primers (GGTA1F-CCAGCAGTATTCTGGGGATAAGA (SEQ ID NO: 44)) and GGTA1R-CCCAGAGGTTACATTTACCCCA3 (SEQ ID NO: 45)). PCR conditions were as follows: 95° C., 3 min; 95° C., 15 s, 60° C., 15 s, and 72° C., 1 s for 40 cycles; and a final extension step of 72° C. for 5 min. PCR fragments were visualised after separation by electrophoresis on 0.8% agarose gels, isolated using a Nucleo-Spin Gel and PCR Clean-up kit (Macherey-Nagel) and sequenced. An overlay of multiple sequences starting around the CRISPR cleavage sites were interpreted as an indication of cleavage activity. The sequences results were then further analysed by Tracking of Indels by DEcomposition (TIDE; Brinkman et al., 2014) to quantify the reduction of the wildtype sequence and the presence of several different sequences with small mutations in the amplified target regions.

4.1.4 Counterselection Against αGal and isolation of Cell Clones

Kidney-derived primary AI pig cells transfected with GGTA1-specific CRISPRs were counter selected as previously described (Fujimura et al., 2008). Briefly, seven days after transfection, cells were harvested and 3×10⁶ cells incubated with 30 μl of biotin-conjugated isolectin IB4 (IB4 lectin from Enzo Life Science, Farmingdale N.Y) in 600 μl of PBS for 15 minutes. Subsequently, the cells were washed in 10 ml PBS to remove unbound isolectin conjugate and incubated with 600 μl streptavidin-conjugated Dynabeads (Invitrogen) on ice, occasionally mixing the cell suspension. Following the incubation, 5 ml PBS was added, the cell suspension transferred to a single well of a six well plate on a plate magnet and incubated for one minute. The IB4 lectin is specific for the αGal epitope; cells that did not bind the Dynabeads remained in the supernatant representing cells that no longer express the αGal epitope. The supernatant was aspirated and the Dynabed selection repeated two more times. Cells from the final supernatant were seeded into a 35 mm culture dish and cultured overnight. Mitotic cells were then manually picked with a glass capillary and individual cells transferred into the wells of eight 96 well plates to isolate cell clones. After approximately 14 days individual cell clones reached confluency and were first transferred to a 48-well plate, then to a 12-well plate and 6-well plate for further expansion. When cell clones reached confluency in a 6-well or alternatively already at the 12-well stage, a small proportion of cells were removed for mutation screening and the remainder of cells cryopreserved to capture the cell clones.

4.1.5 Characterisation of Cell Clones for Site-Specific Mutations and PCR Analysis

Target regions were PCR-amplified with primers GGTA1F/R, visualised and sequenced as described above. All sequencing was done by a commercial service provider (Massey Genome Service, New Zealand).

### 4.2 Results

4.2.1 CRISPR Design and Testing for Cleavage Activity

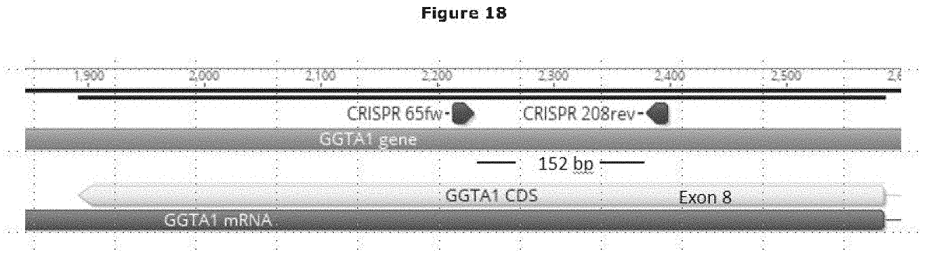

Based on the public pig genome, the CISPOR design tool (Haeussler et al., 2016) was used to identify pairs of CRSIPRs to delete a 152 bp fragment that disrupts the reading frame of the GGTA1 genes in exon 8 (FIG. 18).

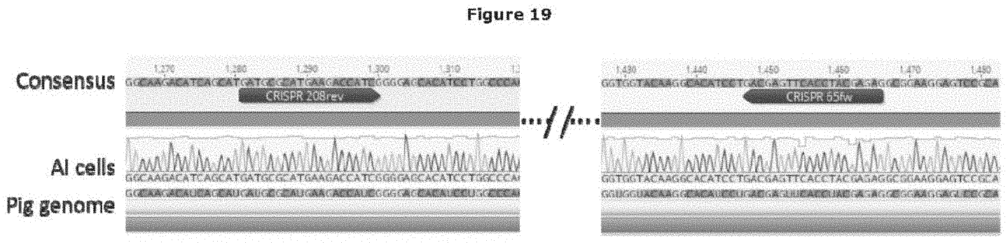

The sequence for the target regions of the GGTA1 gene in the AI pig cells NZK1 was then determined. Comparison of the sequence with the public pig genome revealed the absence of any polymorphisms in the AI pig sequence (FIG. 19). It confirmed the presence of unaltered CRISPR binding sites in AI pig cells and suitability of the two CRISPRs 65fw and 208 rev for editing of the AI pig cells.

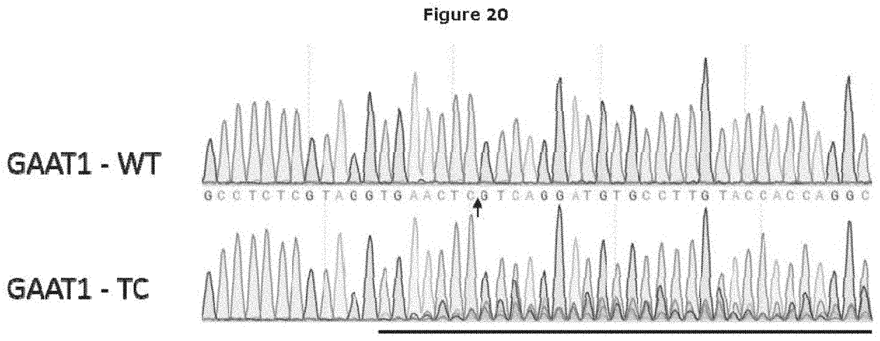

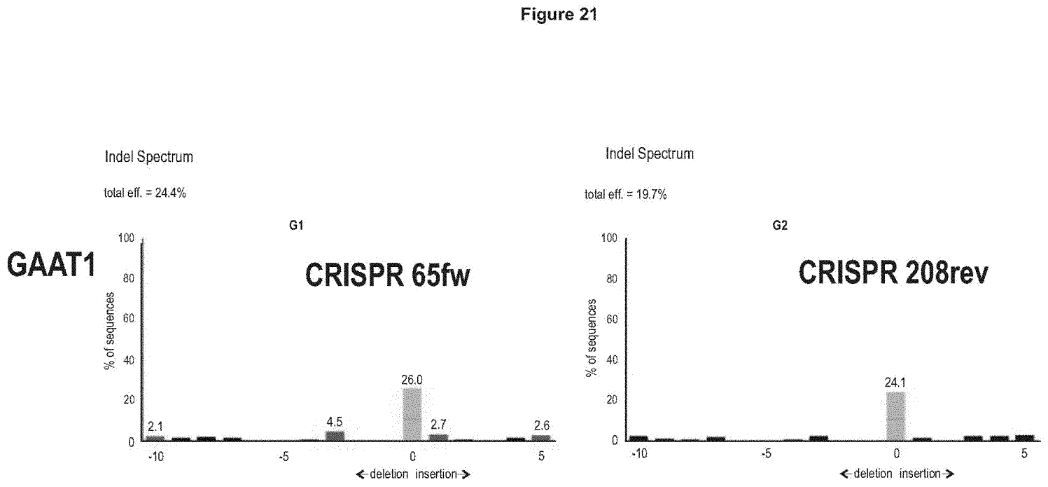

The activity of the CRISPRs were verified by transfecting NZK1 cells with plasmids encoding each of the two CRISPRs and analysing the target locus for editing. Following transfection, cells were harvested and the GGTA1 target region was PCR amplified from their DNA. The sequence analysis of the amplified fragment revealed a unique sequence at the beginning that changed into multiple overlaying sequences from around the predicted CRISPR cleavage site onwards indicating the presence of CRISPR-induced mutations (FIG. 20). This was further confirmed by Tracking of Indels by DEcomposition (TIDE) analysis of the sequences. This detected a strong reduction of the unaltered wildtype sequence from 100% in NZK1 cells to 24.1%-26.0% in the PCR fragments of AI cells transfected with GGTA1-specific CRISPRs. (FIG. 21). In addition, there were new sequence variants comprising small deletions and insertions in close proximity of the CRISPR cleavage site and the presence of several different sequences with small mutations. Taken together, this provided strong evidence for robust, site-specific cleavage activity of the two CRISPRs.

4.2.2 isolation of NZK1 αGal Knockout Cell Clones

NZK1, kidney-derived primary AI pig cells as described in Example 3, were transfected with two plasmids expressing the GGTA1-specific CRISPRs 65fw and 208rev. After seven days of culture, cells were counter selected for the presence of the αGal epitope. From the selected cell population, mitotic cells were manually picked and individually transferred into 96 well plates to isolate cell clones. Following expansion of the cell clones into 12-well or 6-well plates, a small proportion of cells was removed for mutation screening and the remainder of cells cryopreserved to capture the cell clones.

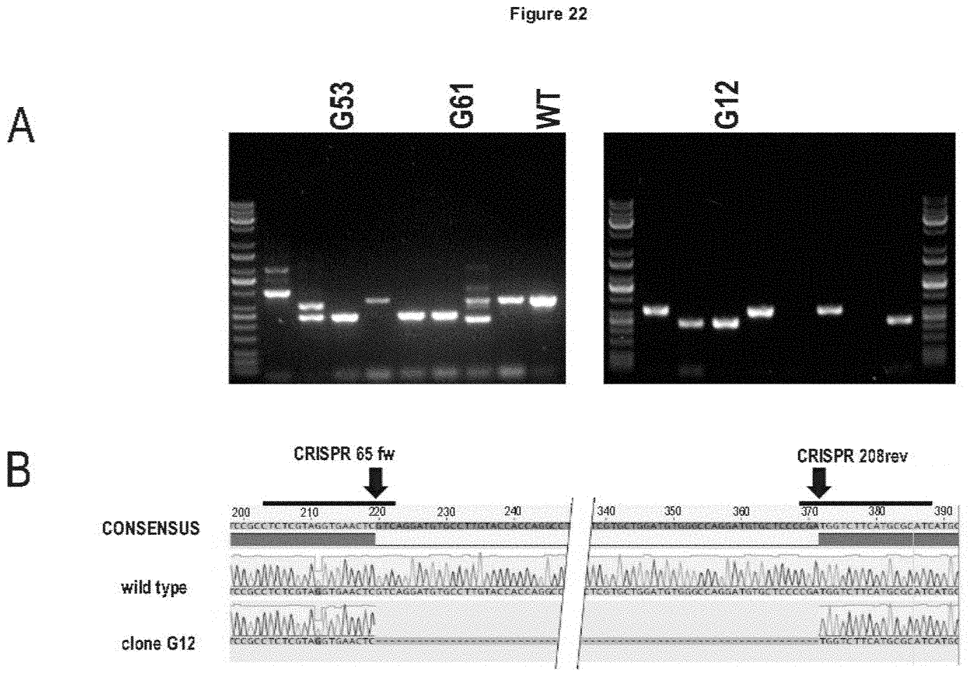

Genomic DNA isolated from the cell clones was PCR amplified for the GGTA1 target region and the size of the amplified fragments analysed on agarose gels (FIG. 22A). This revealed cell clones with two smaller fragments, indicative of the presence of two different deletion alleles while others had just a single fragment of a smaller size (such as cell clones G53, G61 and G12) potentially carrying two alleles with an identical deletion. Cell clones producing a single fragment were sequenced to determine the exact mutations and confirm the presence of two identical deletion alleles (FIG. 22B). All sequenced cell clones had two identical deletion alleles (homozygous, biallelic) with most possessing a 152 bp deletion that correlates with a precise excision of the intervening region between the two predicted CRISPR cleavage sites.

The results for the characterisation of the edited GGTA1 mutations in individual cell clones is summarised in Table 5.

TABLE 5
Summary of characterised GGTA1 knockout cell clones.

| Clone ID | PCR | Sequence | Mutation |
|---|---|---|---|
| G3 | 1 smaller | mixed sequence | N/A |
| | fragment | | |
| G6 | 1 smaller | biallelic deletion | 152 bp deletion |
| | fragment | | |
| G7 | 1 smaller | mixed sequence | N/A |
| | fragment | | |
| G12* | 1 smaller | biallelic deletion | 152 bp deletion |
| | fragment | | |
| G19 | 1 smaller | biallelic deletion | 152 bp deletion |
| | fragment | | |
| G20* | 1 smaller | biallelic deletion | 157 bp deletion |
| | fragment | | |
| G21 | 1 smaller | biallelic deletion | 152 bp deletion |
| | fragment | | |
| G23 | 1 slightly | mixed sequence | N/A |
| | smaller | | |
| | fragment | | |
| G53* | 1 smaller | biallelic deletion | 152 bp deletion |
| | fragment | | |
| G61* | 1 smaller | biallelic deletion | 152 bp deletion |
| | fragment | | |
| G62 | 1 smaller | mixed sequence | N/A |
| | fragment | | |
| G17 | No fragment | N/A | N/A |
| G15 | No fragment | N/A | N/A |

### 4.3 Conclusion

The CRISPR design was effective and produced CRISPRs with site-specific cleavage activity for the target locus in the GGTA1 gene. The use of two GGTA1-specific primers generated many cell clones with a precise excision of the intervening sequence between the predicted cleavage sites of the two CRISPRs. Using this method, GGTA1 knockouts in the NZK1 cell line were generated. This novel cell line could be used for knocking out additional xenoantigens, for expressing human immune-compatible genes, and/or for somatic cell nuclear transfer to generate viable embryos and ultimately animals that could serve as a source of organs, tissues, and cells for xenotransplantation.

### 5. Example 5

This example describes the creation of a line of Auckland Island (AI) pig cells with a functional disruption to the CMAH gene, that lack the Neu5Gc xenoantigen.

### 5.1 Methods

5.1.1 Nucleic Acid Constructs

Two guide RNAs (gRNAs) were designed using the CRISPOR design tool (Haeussler et al., 2016) were designed to target exon 3 of CMAH (68rev-TAAGAATAAGAGCCGCCTGA (SEQ ID NO: 46) and 165fw-TTGAGATTGGCAGCTTCGGC (SEQ ID NO: 47), respectively) beginning at position 19,917,647 and 19,917,702, respectively, of Ensemble gene ENSSSCG00000001099.

DNA oligo nucleotides for both strands of the gRNA were synthesised with short adapters, annealed and cloned into the expression vector pX330 according to published protocols (Ran et al., 2013).

5.1.2 Cell Culture and Transfection

Cell culture and transfection was performed as described in Example 4 section 4.1.2.

5.1.3 CRISPR Activity Assay

The CRISPR activity assay was performed as described in Example 4 section 4.1.3 using the CMAH-specific CRISPRs (68rev, 165fw) and CMAH primers (CMAHF-GAGCTGCCGTAAAGGAGCTT (SEQ ID NO: 48) and CMAHR-CCTTGATGGGTAGGATGGCC3 (SEQ ID NO: 49)).

### 5.2 Results

5.2.1 CRISPR Design and Testing for Cleavage Activity

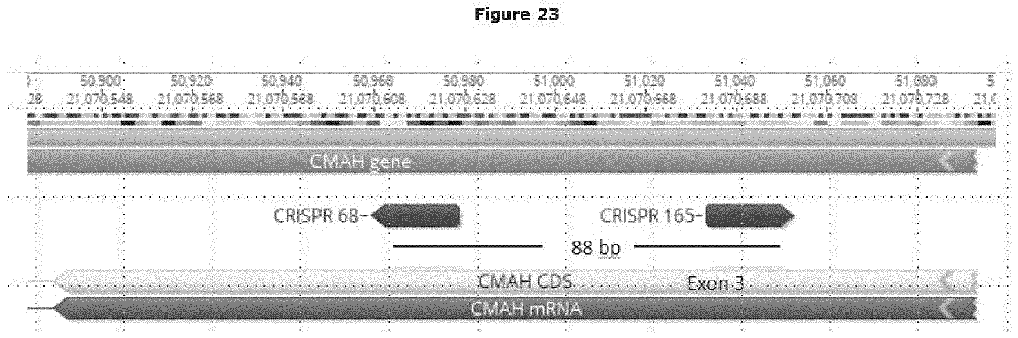

Based on the public pig genome, the CISPOR design tool (Haeussler et al., 2016) Was used to identify pairs of CRSIPRs to delete a 88 bp fragment that disrupts the reading frame of the CMAH gene in exon 3 (FIG. 23).

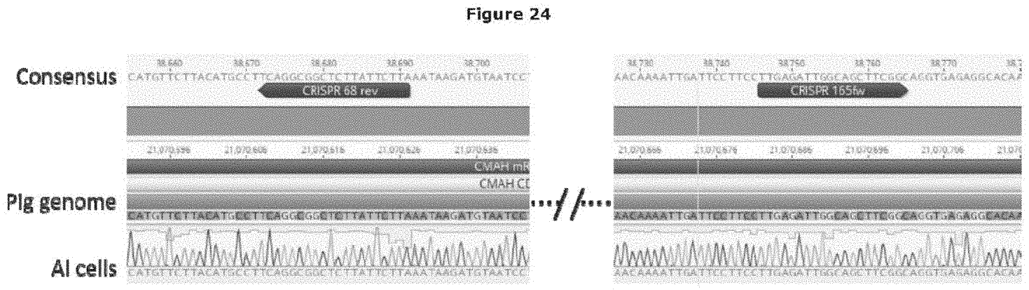

The sequences for the target regions of the CMAH gene in the AI pig cells NZK1 was then determined. Comparison of the sequences with the public pig genome revealed the absence of any polymorphisms in the AI pig sequences (FIG. 24). It confirmed the presence of unaltered CRISPR binding sites in AI pig cells and suitability of the two CRISPRs 68 rev and 165fw for editing of the AI pig cells.

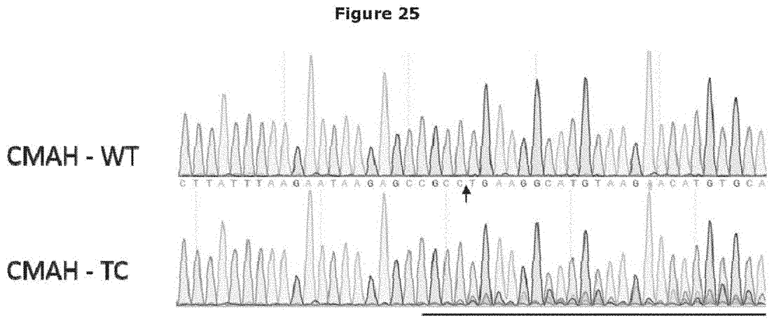

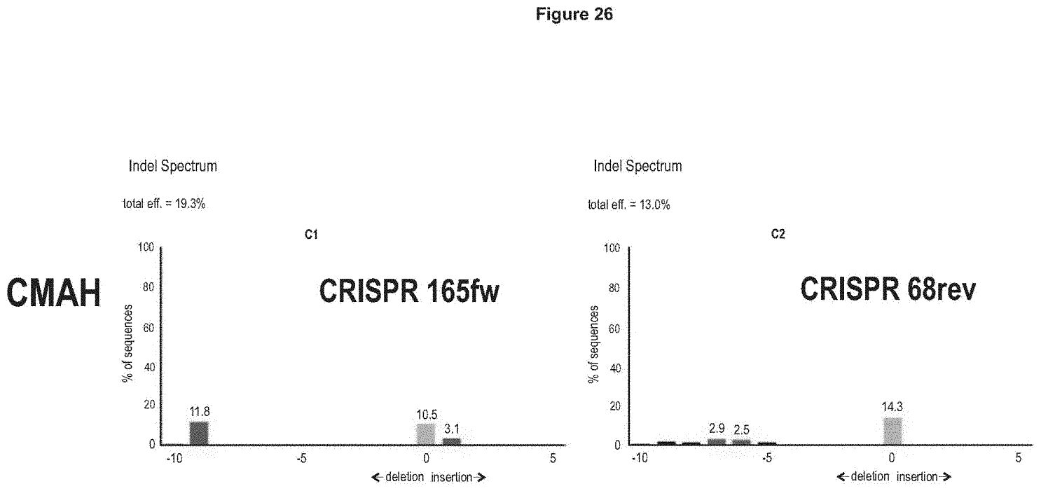

The activity of the CRISPRs were verified by transfecting NZK1 cells with plasmids encoding each of the two CRISPRs and analysing the target locus for editing. Following transfection, cells were harvested and the CMAH target region PCR amplified from their DNA. The sequence analysis of the amplified fragments revealed a unique sequence at the beginning that changed into multiple overlaying sequences from around the predicted CRISPR cleavage site onwards indicating the presence of CRISPR-induced mutations (FIG. 25). This was further confirmed by Tracking of Indels by DEcomposition (TIDE) analysis of the sequences. This detected a strong reduction of the unaltered wildtype sequence from 100% in NZK1 cells to 10.5%-14.3% in the PCR fragments of AI cells transfected with CMAH-specific CRISPRs (FIG. 26). In addition, there were new sequence variants comprising small deletions and insertions in close proximity of the CRISPR cleavage site and the presence of several different sequences with small mutations. Taken together, this provided strong evidence for robust, site-specific cleavage activity of the two CRISPRs.

5.2.2 isolation of NZK3 CMAH Knockout Cell Clones

NZK3, kidney-derived primary AI pig cells as described in Example 3, were transfected with two plasmids expressing the CMAH-specific CRISPRs 165fw and 68rev. After four days of culture, mitotic cells were randomly picked as doublets and individually transferred into 96 well plates to isolate cell clones. Following expansion of the cell clones into 12-well or 6-well plates, a small proportion of cells was removed for mutation screening and the remainder of cells cryopreserved to capture the cell clones.

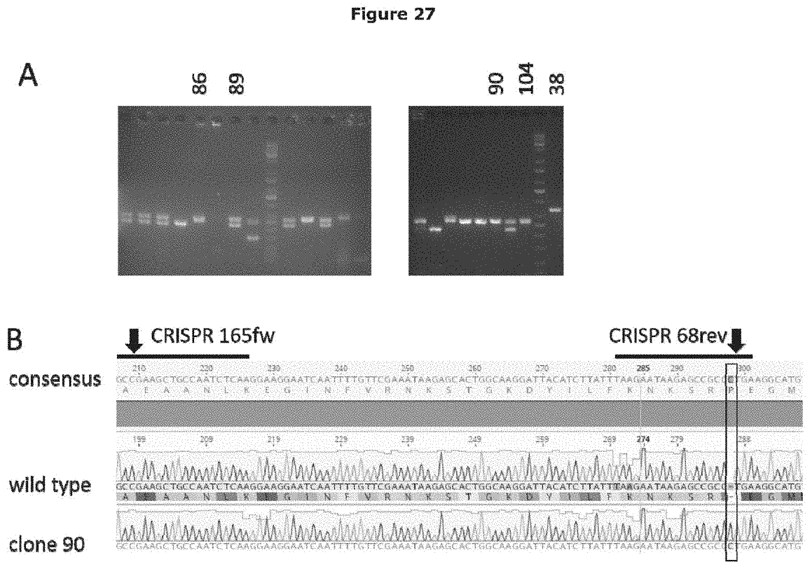

Genomic DNA isolated from 25 candidate cell clones was PCR amplified for the CMAH target region and the size of the amplified fragments analysed on agarose gels (FIG. 27A). All 25 cell clones were subsequently analysed by sequencing (FIG. 27B) and result summarised in Table 6.

TABLE 6
Summary of characterised CMAH knockout NZK3 cell clones.

| Clone ID | PCR | Sequence | Mutation |
|---|---|---|---|
| Several (4) | 1 fragment | Wild type | no |
| Several (13) | 1 or 2 | mixed sequence | 1 or 2 in-frame |
| | fragments | | deletion or WT alleles |
| 20 | 1 fragment | mixed sequence | TBD |
| 34 | 1 fragment | mixed sequence | TBD |
| 38* | 1 fragment | bialielic homozygous | 27 bp plus 164 bp insertion |
| 86* | 1 fragment | biallelic heterozygous | 22 bp deletion; 1 bp deletion |
| 89* | 2 fragments | bialielic heterozygous | 86 bp deletion; 1 bp insertion |
| 90* | 1 fragment | biallelic homozygous | 1 bp insertion |
| 104* | 1 fragment | bialielic heterozygous | 1 bp insertion; 10 bp deletion |
| 105 | 2 fragments | mixed sequence | TBD |

*indicates cell clones assessed as suitable candidates to generate live CMAH knockout pigs.

Of the cell clones analyse, five were deemed suitable for generating CMAH knockout pigs (indicated with * in Table 6). The best candidate was clone 90 which had a homozygous biallelic 1 bp insertion. Another homozygous biallelic cell clone (38) had a 24 bp and a 164 bp insertion at both of the CRISPR cleavage sites. The remaining three cell cllones (86, 89 and 104) were heterozygous biallelic and possessed two different mutant alleles. Several cell clones (4) turned out to be unedited with both genes showing the wild type sequence. Another group of 13 cell clones were not analysed in further detail because they had one remaining wild type allele or possessed one or two in-fame deletion that are unlikely to disrupt the gene function. Other cell clones revealed mixed sequences despite the amplification of what appeared as a single PCR fragment. Our preliminary analysis of the mixed sequences indicated that they are very unlikely suitable candidates and we therefore not further invested any effort into identifying the exact mutations present in these cell clones.

### 5.3 Conclusion

The CRISPR design was effective and produced CRISPRs with site-specific cleavage activity for the target locus in the CMAH gene. Unlike the GGTA1 mutations generated in Example 3, the actual and predicted cleavage sites in the CMAH gene were different, resulting in many cell clones with in-frame deletions and no functional disruption. Further, we observed that the CMAH CRISPR 68rev had a higher activity compared to the second CMAH CRISPPR 165fw.

Overall, the editing efficiency was sufficiently high to allow CMAH KO cell clones to be isolated to produce a novel cell line. Moreover, homozygous biallelic mutants for the isolated cell clone knockouts, could be selected which will facilitate future breeding schemes. This novel cell line could be used for knocking out additional xenoantigens, for expressing human immune-compatible genes, and/or for somatic cell nuclear transfer to generate viable embryos and ultimately animals that could serve as a source of organs, tissues, and cells for xenotransplantation.

### 6. Example 6

This example describes the creation of a line of Auckland Island (AI) pig cells that have functional disruptions to the GGTA1 and CMAH genes, and so lack the αGal and Neu5Gc xenoantigens. This cell line was created from the NZK1 cells described in Example 3.

### 6.1 Methods

6.1.1 CRISPRs

The GGTA1 CRISPRs are as described in Example 4 section 4.2.1 and the CMAH CRISPRs are as described in Example 5 section 5.2.1.

6.1.2 Cell Culture and Transfection

NZK1 cells as described in Example 3 were used. Cell culture and transfection methods are as described in Example 4 section 4.1.2.

6.1.3 Counterselection Against αGal

Counterselection was used as described in Example 4 section 4.1.4.

### 6.2 Results

6.2.1 isolation of NZK1 αGal/CMAH Double Knockout Cell Clones

NZK1 cells were transfected with four plasmids expressing two GGTA1-specific CRISPRs (65fw and 208rev) and two CMAH-specific CRISPRs (165fw and 68rev). After seven days of culture, cells were counter selected for the presence of the αGal epitope based on the assumption that cells with mutations in GGTA1 have a good chance to be also mutated at the CMAH locus and enrich for cells with mutations in both genes. From the selected cell population, mitotic cells were manually picked individually transferred into 96 well plates to isolate cell clones. Following expansion of the cell clones into 12-well or 6-well plates, a small proportion of cells was removed for mutation screening and the remainder of cells cryopreserved to capture the cell clones.

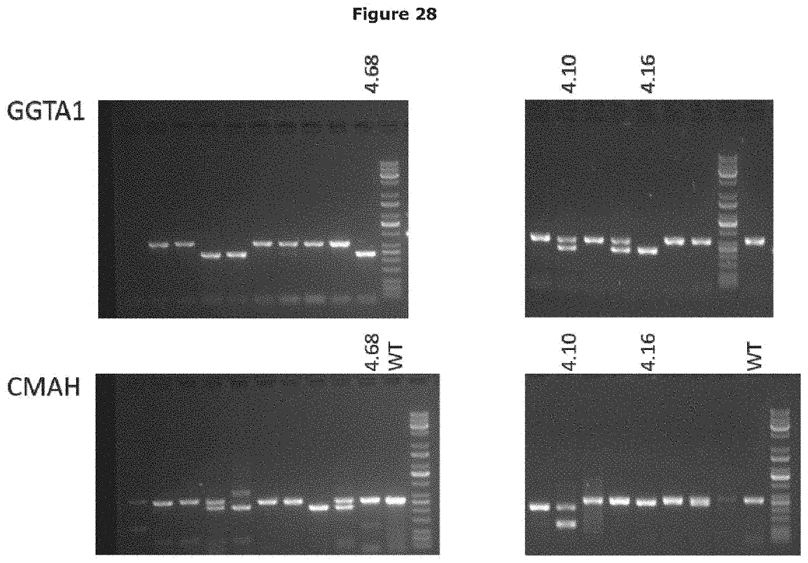

Genomic DNA isolated from the cell clones was separately PCR amplified for the GGTA1 and CMAH target regions using the methods described in Examples 4 and 5. The size of the amplified fragments for both genes was analysed on agarose gels (FIG. 28).

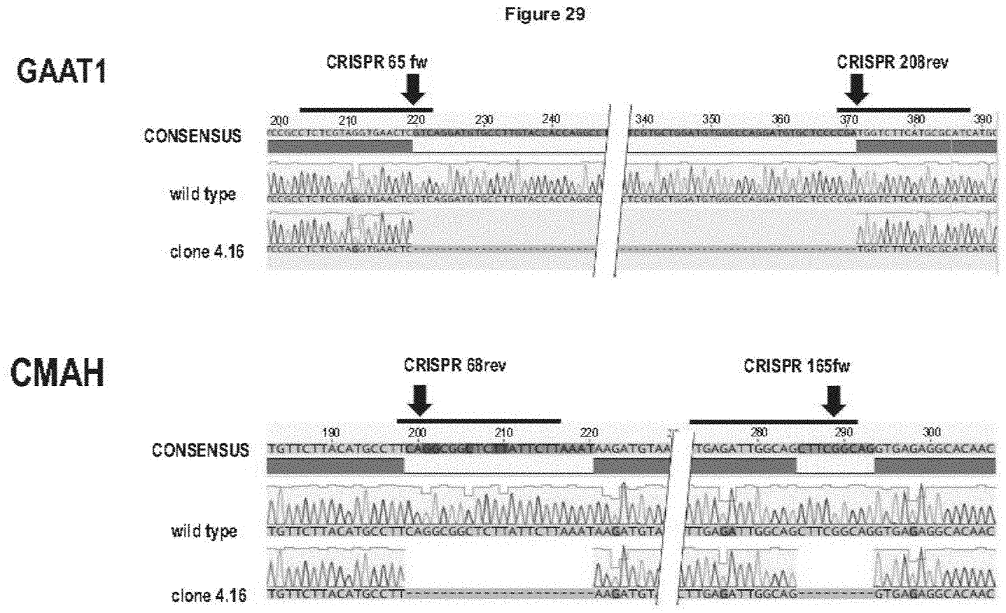

The PCR analysis revealed cell clones with two fragments for one or both genes (e.g. 4.10) but also cell clone with single fragments for GGTA1 and CMAH (e.g. 4.68, 4.16). Of a total of 20 cell clones that were analysed by PCR, 12 were further characterised for their specific mutations by sequence analysis. FIG. 29 depicts the result for cell clone 4.16 revealing a homozygous biallelic 152 bp deletion in the GGTA1 gene and two smaller deletions (9 bp and 22 bp), one each at the two CMAH-specific cleavage sites also present on both alleles (homozygous biallelic). Cell clone 4.68 was another cell clone with homozygous biallelic mutations (152 bp deletion in GGTA1 and 1 bp insertion in CMAH) that disrupts both genes. An additional cell clone (4.5) had disruptions for both genes but for each of the genes it had two different mutant alleles (heterozygous biallelic). A summary of the cell clone characterisation is provided in Table 7.

TABLE 7
Summary of characterised GGTA1 and CMAH
double knockout NZK1 cell clones.

| Clone | GGTA1 PCR | | CMAH PCR | |
|---|---|---|---|---|
| ID | fragments | Mutation | fragments | Mutation |
| 4.1 | 1 (mixed seq.) | N/A | 1 | large del. |
| 4.2 | 1 | 7 bp del. | 1 | N/A |
| | | | (mixed seq.) | |
| 4.3 | 1 (mixed seq.) | N/A | 0 | N/A |
| 4.4 | 2 | N/A | 1 | N/A |
| | | | (mixed seq.) | |
| 4.5* | 2 | 152 bp and | 1 | 1 bp ins./1 bp |
| | | 402 bp del. | (mixed seq.) | conv./6 bp del. |
| | | | | 1 bp ins./5 bp del. |
| 4.10 | 2 | 152 bp and | 2 | 87 bp del. |
| | | 13 bp del. | | 304 bp del. |
| 4.13 | 1 | N/A | 2 | N/A |
| 4.15 | 2 | N/A | 1 (?) | N/A |
| 4.16* | 1 | 152 bp del. | 1 | 9 bp del./22 bp del. |
| 4.18 | 1 | N/A | 1 | 87 bp del. |
| 4.52 | 1 | 2 bp del. | 1 | 87 bp del. |
| 4.68* | 1 | 152 bp del. | 1 | 1 bp ins. |

*indicates cell clones assessed as suitable candidates to generate live GGTA1-CMAH double knockout pigs.

### 6.3 Conclusion

GGAT1 and CMAH double knockouts in the NZK1 cell line were successfully produced, with the help of a counterselection procedure. Moreover, homozygous biallelic mutants were isolated for the isolated cell clone knockout to produce a novel cell line, which will facilitate future breeding schemes. For example, this novel cell line could be used for knocking out additional xenoantigens, for expressing human immune-compatible genes and/or for somatic cell nuclear transfer to generate viable embryos and ultimately animals that could serve as a source of organs, tissues, and cells for xenotransplantation.

### 7. Example 7

This example describes the creation of a line of Auckland Island (AI) pig cells with a functional disruption to the GGTA1 gene, and so lack the αGal xenoantigen. This cell line was created from the NZK3 cells described in Example 3.

### 7.1 Methods

7.1.1 CRISPRs

The GGTA1-specific CRISPRs 65fw and 208rev are as described in Example 4 section 4.2.1.

7.1.2 Cell Culture and Transfection

NZK3, kidney-derived primary AI pig cells as described in Example 3, were transfected with two plasmids expressing the GGTA1-specific CRISPRs 65fw and 208rev. The transfection methods are as described in Example 4 sections 4.1.2.

7.1.1 Counterselection Against αGal

Counterselection was used as described in Example 4 section 4.1.4.

### 7.2 Results

7.2.1 isolation of NZK3 αGal Knockout Cell Clones

After seven days of culture, cells were counter selected for the presence of the αGal epitope. From the selected cell population, mitotic cells were manually picked individually transferred into 96 well plates to isolate cell clones. Following expansion of the cell clones into 12-well or 6-well plates, a small proportion of cells was removed for mutation screening and the remainder of cells cryopreserved to capture the cell clones.

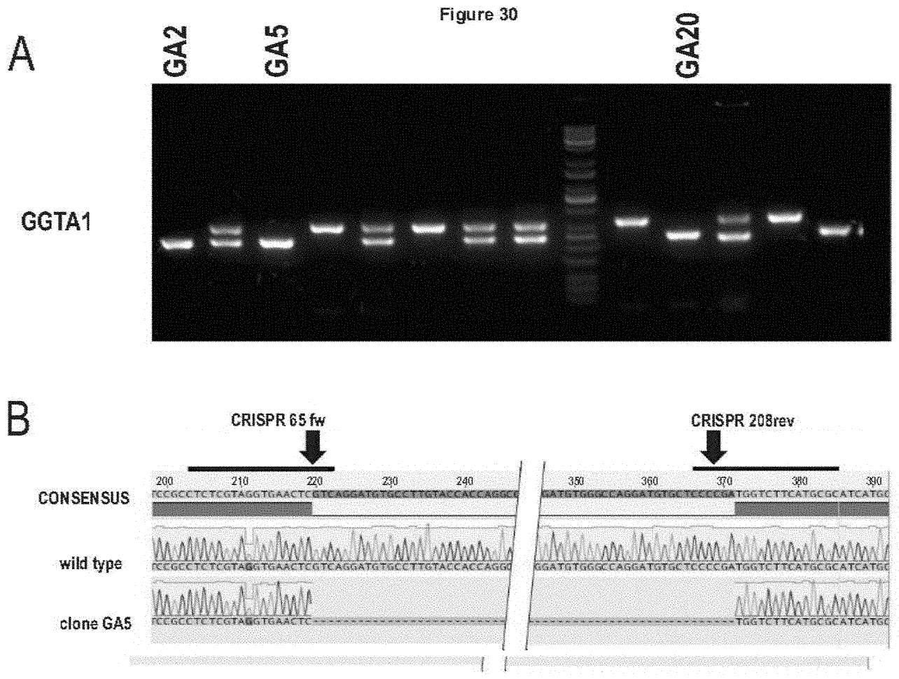

Genomic DNA isolated from the cell clones was PCR amplified for the GGTA1 target region and the size of the amplified fragments analysed on agarose gels (FIG. 30A). Of 28 cell clones we analysed by PCR we identified five cell clones (GA2, GA5, GA20, GA35 and GA65) with a single, smaller fragment, potentially carrying the homozygous biallelic 152 bp deletion alleles we had observed as a frequent editing outcome in our above experiments. Sequencing confirmed that all five cell clones carried the homozygous biallelic 152 bp deletion in the GGTA1 locus (FIG. 30B).

### 7.3 Conclusion

As in Example 3, GGTA1 knockouts were generated, this time in the NZK3 cell line. This novel cell line could be used for knocking out additional xenoantigens, for expressing human immune-compatible genes and/or for somatic cell nuclear transfer to generate viable embryos and ultimately animals that could serve as a source of organs, tissues, and cells for xenotransplantation.

### 8. Example 8

This example describes the creation of a line of Auckland Island (AI) pig cells that have functional disruptions to the GGTA1 and CMAH genes, and so lack the αGal and Neu5Gc xenoantigens. This cell line was created from the NZK3 cells described in Example 3.

### 8.1 Methods

8.1.1 CRISPRs

The GGTA1 CRISPRs are as described in Example 4 section 4.2.1 and the CMAH CRISPRs are as described in Example 5 section 5.2.1.

8.1.2 Cell Culture and Transfection

NZK3, kidney-derived primary AI pig cells as described in Example 3, was transfected with four plasmids expressing two GGTA1-specific CRISPRs (65fw and 208rev) and two CMAH-specific CRISPRs (165fw and 68rev). The transfection methods are as described in Example 4 sections 4.1.2.

8.1.3 Counterselection Against αGal

Counterselection was used as described in Example 4 section 4.1.4.

8.1.4 Fluorescence-Activated Cell Sorting (FACS) Analysis of the αGal Epitope

Cells (1×10⁶) from double KO cell clones were trypsinised, washed with PBS and incubated with FITC conjugated isolectin B4 as previously described (Hauschild et al., 2011). Subsequently, cells were analysed for fluorescence levels on a FACSverse (Becton Dickinson).

### 8.2 Results

8.2.1 isolation of NZK3 αGal/CMAH Double Knockout Cell Clones

After seven days of culture, cells were counter selected for the presence of the αGal epitope and mitotic cells manually picked and individually transferred into 96 well plates to isolate cell clones. Expansion of the cell clones into 12-well or 6-well plates was hampered by contamination of the cultures which limited the number of cell clones to only seven that were available for analysis summarised in Table 8. Only one cell clone GC15 had mutations that disrupted both target genes, although possessing two different mutant alleles for GGTA1 (1 bp insertion plus a 5 bp deletion on one allele and a 152 bp deletion on the other allele) and CMAH (11 bp deletion on one allele and a 107 bp deletion on the other allele). The other six cell clones had in-frame mutations and one or both genes excluding them as candidates for a cell clone with a functional KO of GGTA1 and CMAH.

TABLE 8
Summary of characterised GGTA1 and CMAH
double knockout NZK3 cell clones.

| Clone | GGTA1 PCR | | CMAH PCR | |
|---|---|---|---|---|
| ID | fragments | Mutation | fragments | Mutation |
| GC15* | 2 (hetero) | 1 bp insertion plus | 2 (hetero) | 11 bp deletion; |
| | | 5 bp deletion; | | 107 bp deletion |
| | | 152 bp deletion | | |
| GC16 | 1 (homo) | 36 bp deletion | 1 (homo) | ~290 bp deletion |
| | | (in frame) | | |
| GC18 | 1 (hetero) | 1 bp insertion; | 1 (homo) | 1 bp insertion plus |
| | | 3 bp deletion | | 6 bp deletion |
| | | (in frame) | | |
| GC21 | 2 (hetero) | 1 bp insertion; | 2 (hetero) | 1 bp insertion plus |
| | | 152 bp deletion | | 4 bp deletion; |
| | | | | 87 bp deletion |
| | | | | (in frame) |
| GC24 | 1 (hetero) | Mixed sequence? | 1 (hetero) | 86 bp deletion; |
| | | | | 87 bp deletion |
| | | | | (in frame) |
| GC30 | 1 (homo) | 1 bp insertion plus | 1 (homo) | 87 bp deletion |
| | | 5 bp deletion | | (in frame) |
| GC31 | 2 (hetero) | 136 bp deletion; ? | 2 (hetero) | 210 bp deletion |
| | | | | (in frame); ? |

*indicates cell the cell clone assessed as a suitable candidate to generate live GGTA1-CMAH double knockout pigs.

Because of the availability of a single cell clone with only heterozygous biallelic mutations for both genes that would complicate subsequent breeding programmes due to the random segregation of the different alleles, we decided to repeat the transfection and isolate additional KO cell clones. For this repeat, we transfected NZK3 with three plasmids expressing two GGTA1-specific CRISPRs (65fw and 208rev) and a single CMAH-specific CRISPRs (68rev). After seven days of culture, cells were counter selected for the presence of the αGal epitope and mitotic cells were manually picked and individually transferred into 96 well plates to isolate cell clones. Following expansion of the cell clones into 12-well or 6-well plates, a small proportion of cells was removed for mutation screening and the remainder of cells cryopreserved to capture the cell clones.

Genomic DNA isolated from 52 cell clones was separately PCR amplified for the GGTA1 and CMAH target regions and target regions sequenced for 21 clones to determine the precise sequence change at the target loci. The analysis demonstrated that we had isolated four cell clones that had homozygous biallelic mutations, disrupting the reading frames for the GGTA1 and the CMAH gene (Table 9). All five cell clones are suitable candidates for the generation of double KO AI pigs.

TABLE 9
Double knockout cell clones with homozygous
knockout mutant alleles for GGTA1 and CMAH.

| Clone | GGTA1 PCR | | CMAH PCR | |
|---|---|---|---|---|
| ID | fragments | Mutation | fragments | Mutation |
| 3CG61 | 1 (homo) | 152 bp deletion | 1 (homo) | 1 bp deletion |
| 3CG72 | 1 (homo) | 22 bp deletion | 1 (homo) | 1 bp deletion |
| 3CG27 | 1 (homo) | 154 bp deletion | 1 (homo) | 1 bp insertion |
| 3CG12 | 1 (homo) | 152 bp deletion | 1 (homo) | 71 bp deletion |

8.2.2 Confirmation of the Functional Disruption of the GGTA1 Gene

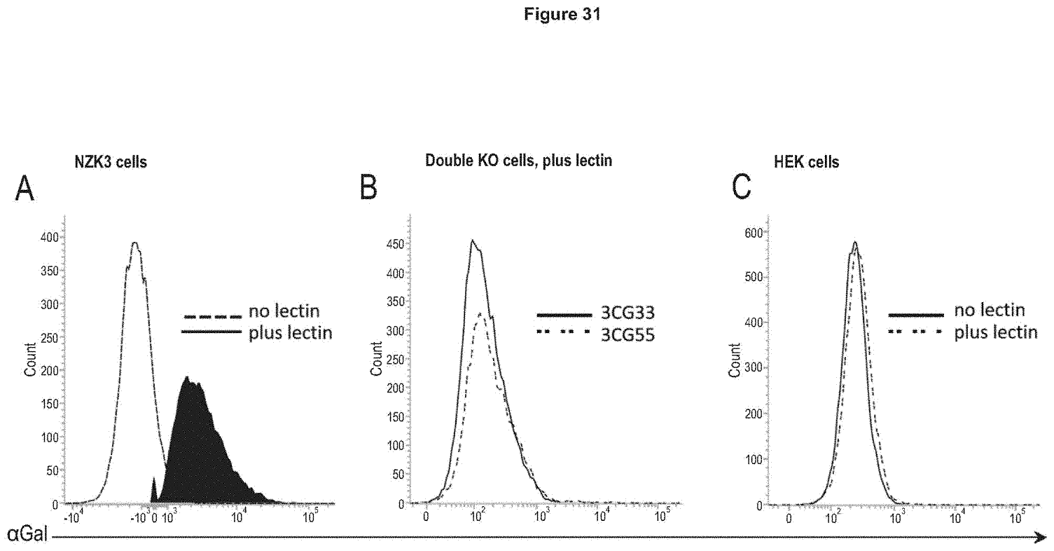

The use of two CRISPRs for the disruption of the GGTA1 gene resulted in most of our cell clones having a precise excision of a 152 bp fragment in exon 8. To verify that this mutation functionally disrupts the GGTA1 gene we have analysed cell from two double KO cell clones (3CG33, 3CG55) with a homozygous, biallelic 152 bp deletion in GGTA1. The cells were incubated with an isolectin B4-FITC conjugate, that specifically binds the αGal epitope. After removal of any non-bound isolectin conjugate, cells were analysed by flowcytometry for the binding or non-binding of the fluorescently-labelled isolectin as an indication of the presence or absence of the αGal epitope. In the absence of isolectin conjugate, wild type AI pig cells are non-fluorescent. Incubated with isolectin the cells become fluorescent indicated by a strong shift in the fluorescence intensity due to the binding of the isolectin conjugate to the αGal epitope on the surface of the AI wild type cells (FIG. 31A). In contrast, human HEK cells which do not express the αGal epitope remain non-fluorescent independent of the incubation with the isolectin conjugate (FIG. 31C). Double KO cells with a 152 bp deletion in exon 8 of GGTA1 remain non-fluorescent in the presence of the isolectin conjugate and show the same pattern as human HEK cells, demonstrating the double KO AI pig cells no longer express the αGal epitope (FIG. 31B).

### 8.3 Conclusion

Using CRISPR, GGAT1 and CMAH double knockouts were generated in the NZK3 cell line with the help of a counterselection procedure to produce a novel cell line.

The predominant mutation in these GGTA1 KO cell clones was a 152 bp deletion in exon 8. Using flow cytometry, it was demonstrated that this mutation functionally disrupts the GGTA1 gene and results in AI pig cells that no longer express the αGal epitope. However, it was not possible to test for the functional disruption of CMAH because CMAH KO cells can take up and incorporate the CMAH-specific glycosylation Neu5Gc from the culture medium. Testing will become feasible once tissue or blood samples from CMAH KO fetuses and piglets are available. This novel cell line could be used for knocking out additional xenoantigens, for expressing human compatible genes and/or for somatic cell nuclear transfer to generate viable embryos and ultimately animals that could serve as a source of organs, tissues, and cells for xenotransplantation.

*All gene editing was carried out under contract by AgResearch Limited, New Zealand.

### 9. Example 9

This example describes the development of porcine somatic cell nuclear transfer (SCNT) for generating blastocysts from gene-edited cell lines.

### 9.1 Methods

Ovaries were sourced from pre-pubertal gilts slaughtered at the Ruakura abattoir (AgResearch, New Zealand). Cumulus-oocyte complexes (COCs) were recovered by follicular aspiration and matured in vitro (IVM) for 44 h. Two IVM systems to mature porcine oocytes were compared: biphasic (ESOF and LSOF) IVM1/2 medium containing 1 mM dibutyryl cyclic adenosine monophosphate for the first 20-24 h (Bagg et al., 2006; Somfai and Hirao, 2011) and FLI medium supplemented with FGF2, LIF and IGF1 for the entire IVM period (Yuan et al., 2017).

COCs were denuded by vortexing with 0.1% hyaluronidase (Sigma) at 2000 rpm for about 60-90 sec. Oocytes were washed twice in HEPES-buffered TCM199 medium (H199)+10% FCS. Denuded oocytes were incubated for 10 min with 0.2M sucrose in H199+10% FCS. Oocytes that maintained a regular, round shape (but smaller diameter) along with a first polar body (1PB) were selected for use. Those oocytes that displayed a very irregular shape after sucrose treatment were discarded, as these are of poorer developmental quality (Dang-Nguyen et al., 2018; Lee et al., 2014).

For zona-free SCNT, pronase was used to remove the zona pellucida. Oocytes were washed thoroughly before staining DNA with 5 μg/mL H33342 plus 7.5 μg/mL cytochalasin B (all Sigma) for 5 min. A 24 μm outer diameter blunt-ended pipette was used for UV-assisted enucleation of metaphase II stage oocytes at 35° C.

Donor cells (e.g. NZK1 kidney fibroblasts) were trypsinised to a single cell suspension after 5-7 days in medium with 0.5% FCS. Single cells were adhered to each enucleated cytoplast with the aid of 20 μg/mL phytohemagglutinin in H199+3 mg/mL BSA. Electrical fusion was achieved with two direct current pulses of either 1.2 kV/cm for 30 μs or 2 kV/cm for 10 μs in fusion buffer without calcium at approximately 48 h after the start of IVM. Following fusion, reconstructed embryos were held in embryo culture medium without Ca+10% FCS+5 μg/mL cytochalasin B at 38.5° C. in 5% CO₂ in air for 2-4 h before activation.

To maintain high levels of maturation promoting factor-cyclin B complex in oocytes aiding nuclear reprogramming, some experiments compared the addition of 5 μM of the proteasome inhibitor MG132 (Sigma). This was added to media formulations from the time of sucrose selection of oocytes until activation of NT reconstructed embryos (Le Bourhis et al., 2010).

Activation of reconstructed NT embryos was initiated with three direct current pulses of 1.0-1.5 kV/cm for 80 μs each, in buffer containing 0.1 mM calcium, ˜50-52 h after the start of IVM.

Within 5 min of electrical stimulation, reconstructed embryos were transferred to culture medium (e.g. ESOF) supplemented with 2 mM 6-dimethylaminopurine+5 μg/mL cytochalasin B and cultured individually in 5 μL drops for 3 h at 38.5° C. In 5% CO₂. Afterwards, embryos were washed thoroughly in HEPES SOF before in vitro culture for 6-7 days. Approximately 10-12 zona-free embryos were group-cultured in microwell depressions made in 20 μL drops of ESOF (or alternative medium) for four days in a low oxygen atmosphere (5:7:88, CO₂:O₂:N₂) in a modular incubator at 38.5° C. Media formulation was changed to LSOF (or alternative medium) from Day 4 to 7. On Day 6, any late morula and early blastocyst-stage embryos were transferred individually to 5 μL drops of LSOF (+5% FCS if required) under oil and culture continued. Embryo development was assessed on Day 6 or 7 and the numbers of nuclei in resulting blastocysts determined in selected experiments.

### 9.2 Results

The FLI media system resulted in significantly more usable oocytes after IVM compared to IVM1/2. However, development of SCNT embryos, but not parthenogenetically activated oocytes (PG), was three-fold greater with the biphasic IVM system (Table 10). Zona-free embryo development of SCNT embryos was substantially less compared to the PG controls.

TABLE 10
Comparison of two IVM systems for the development of porcine
embryos derived from either somatic cell nuclear transfer
(SCNT) or parthenogenetic oocyte activation (PG).

| | | Round | | | | | Blastocyst |
|---|---|---|---|---|---|---|---|
| IVM | | cytoplasm + | Embryo | | | | Development |
| system | nIVM | 1PB | type | Fusion | nIVC | Cleavage | Day 7 |
| IVM1/2 | 1231 | 52%^{a} | SCNT | 77% | 199 | 93% | 6%^{c} |
| | | | PG | n/a | 247 | 94% | 14% |
| FLI | 1271 | 63%^{b} | SCNT | 73% | 288 | 92% | 2%^{d} |
| | | | PG | n/a | 300 | 96% | 17% |

nIVM = number of oocytes matured in vitro. 1PB = first polar body in oocytes. nIVC = number of embryos cultured in vitro.
^{ab}P < 0.001.
^{cd}P < 0.05

A preliminary experiment examining the addition of 5 μM of the proteasome inhibitor MG132 between oocyte selection and activation of NT reconstructs was unable to show any benefit in terms of in vitro development following SCNT or PG (Table 11).

TABLE 11
The addition of the proteasome inhibitor MG132 prior
to activation did not increase porcine embryo development
following somatic cell nuclear transfer (SCNT) or
parthenogenetic oocyte activation (PG).

| Media | Embryo | | | | Blastocyst |
|---|---|---|---|---|---|
| system | type | Fusion | nIVC | Cleavage | Development Day 7 |
| Control | SCNT | 37% | 57 | 93% | 13%^{a} |
| | PG | n/a | 96 | 100% | 23% |
| MG132 | SCNT | 49% | 81 | 99% | 11%^{b} |
| | PG | n/a | 102 | 100% | 23% |

nIVC = number of embryos cultured in vitro.
^{ab}P < 0.05.

Focus was on developing a zona-free SCNT procedure in pig as experience in other species has demonstrated advantages in simplifying embryo reconstruction and increasing laboratory throughput. However, rates of embryo development are still lower in our current zona-free in vitro culture system in pig compared to more conventional zona-intact procedures, as demonstrated with parthenogenetically activated porcine oocytes (Table 12).

TABLE 12
Greater in vitro embryo development with zona-intact compared
to zona-free parthenogenetically activated oocytes (PG).

| | | | Blastocyst |
|---|---|---|---|
| PGs | nIVC | Cleavage | Development Day 7 |
| Zona-intact | 344 | 96% | 20%^{a} |
| Zona-free | 203 | 94% | 7%^{b} |

nIV = number embryos cultured in vitro.
^{ab}P < 0.0001.

### 9.3 Conclusion

It is known that the development of zona-free porcine SCNT embryos to the blastocyst-stage on days 6-7 of in vitro culture increased from 5% to 15% by selecting better quality oocytes after treatment with sucrose (Dang-Nguyen et al., 2018). Despite using this oocyte selection procedure here, development results following zona-free SCNT were generally low and variable, especially compared to parthenogenetically activated oocyte controls. The in vitro maturation of oocytes in a biphasic system over a 44-hour period, incorporating the use of dbcAMP, increased the rates of blastocyst development of SCNT embryos compared to the FLI oocyte maturation system (6% vs. 2%).

Calcium-free cell fusion buffer was used to prevent premature oocyte activation which is an obstacle to reprogramming a differentiated nucleus following SCNT. Preliminary investigations with MG132 addition to maintain maturation promoting factor-cyclin B complex, to facilitate epigenetic reprogramming of donor nuclei and improve SCNT embryo development, showed no benefit at the concentration and time period examined here.

A zona-free SCNT protocol in pig was initially favoured as in other species this is technically easier and results in greater throughput compared to more conventional zona-intact methods. However, unlike the situation in cattle, the present zona-free embryo culture system in pig still compromises in vitro developmental potential compared to a zona-intact embryo culture system. Thus, preliminary experiments have been carried out producing zona-intact SCNT pig blastocysts utilizing electrode needles to improve cell fusion efficiencies. To improve the success of in vivo development of porcine SCNT embryos in future work, the current recommendation is to transfer reconstructed 1-cell embryos to the oviducts of recipient gilts rather than in vitro blastocysts transferred to the uteri. To manage the logistics of embryo transfer this may require embryo vitrification.

The present inventors contemplate using the blastocysts produced by the methods described herein, having one or more xenoantigen knockouts, to be implanted into female PERV-C and PERV-A/C negative foundation pigs selected by the CGS method of the invention, to produce a novel cloned PERV-C, PERV-A/C and xenoantigen negative pig strain (designated NZeno-1) as donors for xenotransplantation.

### REFERENCES

Bagg, M. A., Nottle, M. B., Grupen, C. G., Armstrong, D. T., 2006. Effect of dibutyryl cAMP on the cAMP content, meiotic progression, and developmental potential of in vitro matured pre-pubertal and adult pig oocytes. Mol. Reprod. Dev. 73, 1326-1332. https://doi.org/10.1002/mrd.20555
Brinkman, E. K., Chen, T., Amendola, M., van Steensel, B., 2014. Easy quantitative assessment of genome editing by sequence trace decomposition. Nucleic Acids Res. 42, e168-e168. https://doi.org/10.1093/nar/gku936
Dang-Nguyen, T. Q., Nguyen, H. T., Somfal, T., Wells, D., Men, N. T., Viet-Linh, N., Noguchi, J., Kaneko, H., Kikuchi, K., Nagai, T., 2018. Sucrose assists selection of high-quality oocytes in pigs. Anim. Sci. J. 89, 880-887. https://doi.org/10.1111/asj.13015
Fujimura, T., Takahagi, Y., Shigehisa, T., Nagashima, H., Miyagawa, S., Shirakura, R., Murakami, H., 2008. Production of alpha 1,3-Galactosyltransferase gene-deficient pigs by somatic cell nuclear transfer: A novel selection method for gal alpha 1,3-Gal antigen-deficient cells. Mol. Reprod. Dev. 75, 1372-1378. https://doi.org/10.1002/mrd.20890
Garkavenko, O., Wynyard, S., Nathu, D., Muzina, M., Muzina, Z., Scobie, L., Hector, R. D., Croxson, M. C., Tan, P., Elliott, B. R., 2008. Porcine Endogenous Retrovirus Transmission Characteristics From a Designated Pathogen-Free Herd. Transplant. Proc. 40, 590-593. https://doi.org/10.1016/j.transproceed.2008.01.051
Haeussler, M., Schönig, K., Eckert, H., Eschstruth, A., Mianné, J., Renaud, J.-B., Schneider-Maunoury, S., Shkumatava, A., Teboul, L., Kent, J., Joly, J.-S., Concordet, J.-P., 2016. Evaluation of off-target and on-target scoring algorithms and integration into the guide RNA selection tool CRISPOR. Genome Biol. 17, 148. https://doi.org/10.1186/s13059-016-1012-2
Kimsa, Magdalena, Strzalka-Mrozik, B., Kimsa, Malgorzata, Gola, J., Nicholson, P., Lopata, K., Mazurek, U., 2014. Porcine Endogenous Retroviruses in Xenotransplantation-Molecular Aspects. Viruses 6, 2062-2083. https://doi.org/10.3390/v6052062
Kurome, M., Kessler, B., Wuensch, A., Nagashima, H., Wolf, E., 2015. Nuclear transfer and transgenesis in the pig. Methods Mol. Biol. Clifton N.J. 1222, 37-59. https://doi.org/10.1007/978-1-4939-1594-1_4
Le Bourhis, D., Beaujean, N., Ruffini, S., Vignon, X., Gall, L., 2010. Nuclear Remodeling in Bovine Somatic Cell Nuclear Transfer Embryos Using MG132-Treated Recipient Oocytes. Cell. Reprogramming 12, 729-738. https://doi.org/10.1089/cell.2010.0035
Lee, J., Lee, Y., Park, B., Elahi, F., Jeon, Y., Hyun, S.-H., Lee, E., 2014. Developmental competence of IVM pig oocytes after SCNT in relation to the shrinkage pattern induced by hyperosmotic treatment. Theriogenology 81, 974-981. https://doi.org/10.1016/j.theriogenology.2014.01.022
Niu, D., Wei, H.-J., Lin, L., George, H., Wang, T., Lee, I.-H., Zhao, H.-Y., Wang, Y., Kan, Y., Shrock, E., Lesha, E., Wang, G., Luo, Y., Qing, Y., Jiao, D., Zhao, H., Zhou, X., Wang, S., Wei, H., Güell, M., Church, G. M., Yang, L., 2017. Inactivation of porcine endogenous retrovirus in pigs using CRISPR-Cas9. Science 357, 1303-1307. https://doi.org/10.1126/science.aan4187
Ran, F. A., Hsu, P. D., Wright, J., Agarwala, V., Scott, D. A., Zhang, F., 2013. Genome engineering using the CRISPR-Cas9 system. Nat. Protoc. 8, 2281-2308. https://doi.org/10.1038/nprot.2013.143
Richter, A., Kurome, M., Kessler, B., Zakhartchenko, V., Klymiuk, N., Nagashima, H., Wolf, E., Wuensch, A., 2012. Potential of primary kidney cells for somatic cell nuclear transfer mediated transgenesis in pig. BMC Biotechnol. 12, 84. https://doi.org/10.1186/1472-6750-12-84
Somfai, T., Hirao, Y., 2011. Synchronization of In Vitro Maturation in Porcine Oocytes, in: Banfalvi, G. (Ed.), Cell Cycle Synchronization: Methods and Protocols, Methods in Molecular Biology. Humana Press, Totowa, N.J., pp. 211-225. https://doi.org/10.1007/978-1-61779-182-6_14
Tang, H.-B., Ouyang, K., Rao, G.-B., Ma, L., Zhong, H., Bal, A., Qin, S., Chen, F., Lin, J., Cao, Y., Liao, Y.-J., Zhang, J., Wu, J., 2016. Characterization of Complete Genome Sequences of a Porcine Endogenous Retrovirus isolated From China Bama Minipig Reveals an Evolutionary Time Earlier Than That of isolates From European Minipigs. Transplant. Proc. 48, 222-228. https://doi.org/10.1016/j.transproceed.2015.12.005
Venter, J. C., 1998. GENOMICS: Shotgun Sequencing of the Human Genome. Science 280, 1540-1542. https://doi.org/10.1126/science.280.5369.1540
Weintraub, K., 2019. A CRISPR startup is testing pig organs in monkeys to see if they're safe for us [WWW Document]. MIT Technol. Rev. URL https://www.technologyreview.com/s/613666/crispr-pig-organs-are-being-implanted-in-monkeys-to-see-if-theyre-safe-for-humans/ (accessed 7.26.19).
Xiang, S., Ma, Y., Yan, Q., Lv, M., Zhao, X., Yin, H., Zhang, N., Jia, J., Yu, R., Zhang, J., 2013. Construction and characterization of an infectious replication competent clone of porcine endogenous retrovirus from Chinese miniature pigs. Virol. J. 10, 228. https://doi.org/10.1186/1743-422X-10-228
Yang, L., Güell, M., Niu, D., George, H., Lesha, E., Grishin, D., Aach, J., Shrock, E., Xu, W., Poci, J., Cortazio, R., Wilkinson, R. A., Fishman, J. A., Church, G., 2015. Genome-wide inactivation of porcine endogenous retroviruses (PERVs). Science 350, 1101-1104. https://doi.org/10.1126/science.aad1191
Yuan, Y., Spate, L. D., Redel, B. K., Tian, Y., Zhou, J., Prather, R. S., Roberts, R. M., 2017. Quadrupling efficiency in production of genetically modified pigs through improved oocyte maturation. Proc. Natl. Acad. Sci. 114, E5796-E5804. https://doi.org/10.1073/pnas.1703998114

1. A method of selecting pigs suitable as donors for xenotransplantation of tissues and/or cells, or as foundation pigs for further manipulation, said method comprising the step:
a) providing a designated pathogen-free (DPF) pig herd having a low porcine endogenous retrovirus (PERV) copy number of between 4 and 40;
b) testing the PERV status of individual pigs of the herd using complete genome sequencing (CGS);
c) identifying individual pigs that have PERV-C and PERV-A/C negative status; and
d) selecting said PERV-C and PERV-A/C negative pigs as donor pigs for xenotransplantation, or as foundation pigs for further manipulation.

2. The method of claim 1, further comprising identifying the PERV-A and PERV-B status of the PERV-C negative pigs of step d) and further selecting pigs that have a very low number of functioning PERV-A and PERV-B sequences of between 1-10.

3. The method of claim 1, wherein the pigs are Auckland Island (AI) pigs.

4. The method of claim 1, wherein the pigs are PERV-null.

5. A pig selected by the method of claim 1.

6. A method of breeding a herd of pigs that have no functional PERV genomic sequences suitable as donors for xenotransplantation, or as foundation pigs for further manipulation, said method comprising the steps:
a) selecting PERV-C and PERV-A/C negative male and female pigs using the CGS selection method of claim 1;
b) analysing the chromosomal location of any functioning PERV-A and PERV-B gene sequences in the male pigs of step a);
c) selecting male pigs that have functioning PERV-A and/or PERV-B present in the Y-chromosome only;
d) breeding the male pigs of step c) with the female pigs of step a) to produce progeny; and
e) selecting female progeny that will lack the paternal functional PERV-A and/or PERV-B as future breeding stock to produce a herd of pigs that have no functional PERV genomic sequences and that are suitable as donors of tissues and/or cells for xenotransplantation, or as foundation pigs for further manipulation.

7. A pig herd bred by the method of claim 6.

8. A method of providing donor pigs suitable for xenotransplantation of whole organs, tissues and/or cells into a human recipient comprising the steps:
(a) selecting PERV-C and PERV-A/C negative donor pigs using the CGS method of claim 1,
(b) establishing a PERV-C and PERV-A/C negative cell line from the pigs of step (a);
(c) gene editing cells of said cells of step (b) to eliminate or deactivate one or more xenoantigen genes;
(d) optionally gene editing selected isolated cells of step (c) to express one or more human genes selected from A20, CD39, CD46, CD47, CD55, CD59, hemoxygenase-1 (HO-1), HLA-E, HLA-G, thrombomodulin (TM), CTLA4-Ig, and LEA29Y;
(e) establishing a gene-edited cell line from the pig cells of step (c) or (d);
(f) carrying out somatic cell nuclear transfer from one or more of said gene edited cells of step (e) into an oocyte from a PERV-C and PERV-A/C negative pig in vitro;
(g) culturing to blastocyst stage;
(h) transferring said blastocyst(s) to a female PERV-C and PERV-A/C negative pig and growing to full term; and
(i) providing donor pigs that are PERV-C and PERV-A/C negative and gene edited for one or more xenoantigens.

9. The method of claim 8, wherein the one or more xenoantigen genes is selected from GGTA1, CAMH, B4GalNT2, Neu5Gc, ASGR1 and SLA.

10. A pig cell line that is PERV-C and PERV-A/C negative and gene edited for inactivation or deletion of one or more xenoantigens and optionally for insertion of one or more human genes, produced by the method of steps (a)-(d) of claim 8.

11. Donor pigs that are PERV-C and PERV-A/C negative and gene edited for inactivation or deletion of one or more xenoantigens and optionally gene edited for expression of one or more human genes, produced by the method of claim 8.

12. Organs, tissues and/or cells from the pigs of claim 11 for use in xenotransplantation.

13. The organs, tissues and/or cells for use in xenotransplantation of claim 12, selected from the group consisting of kidney, liver, lung, heart, brain, pancreas, muscle, blood, bone, testes and ovary.

14. The tissue and/or cells for use in xenotransplantation of claim 12, selected from pancreatic islets, hepatocytes, non-parenchymal liver cells, gall bladder epithelial cells, gall bladder endothelial cells, bile duct epithelial cells, bile duct endothelial cells, hepatic vessel epithelial cells, hepatic vessel endothelial cells, sinusoid cells, choroid plexus cells, fibroblasts, Sertoli cells, adrenal chromaffin cells and muscle cells.

15. A method of treating a patient suffering from or predisposed to a disease, disorder or condition associated with a deficiency in or absence of organ function, said method comprising transplanting an organ, tissue and/or cells of claim 12 to a patient in need thereof.

16. The method of claim 2, wherein the selecting step is selecting pigs that have a very low number of functioning PERV-A and PERV-B sequences of between 1-5.

17. The method of claim 2, wherein the selecting step is selecting pigs that have a very low number of functioning PERV-A and PERV-B sequences of between 1-2.


SEQUENCE LISTING (49 sequences)

SEQ ID NO: 1 - DNA, 55 bp, Sus scrofa
ggcaagacatcagcatgatgcgcatgaagaccatcggggagcacatcctggccca

SEQ ID NO: 2 - DNA, 56 bp, Sus scrofa
ggtggtacaaggcacatcctgacgagttcacctacgagaggcggaaggagtccgca

SEQ ID NO: 3 - DNA, 55 bp, Sus scrofa; identical to SEQ ID NO: 1

SEQ ID NO: 4 - DNA, 56 bp, Sus scrofa; identical to SEQ ID NO: 2

SEQ ID NO: 5 - RNA, 55 nt, Sus scrofa
ggcaagacaucagcaugaugcgcaugaagaccaucggggagcacauccuggccca

SEQ ID NO: 6 - RNA, 56 nt, Sus scrofa
ggugguacaaggcacauccugacgaguucaccuacgagaggcggaaggaguccgca

SEQ ID NO: 7 - DNA, 46 bp, Sus scrofa
gcctctcgtaggtgaactcgtcaggatgtgccttgtaccaccaggc

SEQ ID NO: 8 - DNA, 50 bp, Sus scrofa
ccgcctctcgtaggtgaactcgtcaggatgtgccttgtaccaccaggcct

SEQ ID NO: 9 - DNA, 54 bp, Sus scrofa
tcgtgctggatgtgggccaggatgtgctccccgatggtcttcatgcgcatcatg

SEQ ID NO: 10 - DNA, 49 bp, Sus scrofa
ccgcctctcgtaggtgaactcgtcaggatgtgccttgtaccaccaggcc

SEQ ID NO: 11 - DNA, 55 bp, Sus scrofa
ctcgtgctggatgtgggccaggatgtgctccccgatggtcttcatgcgcatcatg

SEQ ID NO: 12 - DNA, 21 bp, Sus scrofa
ccgcctctcgtaggtgaactc

SEQ ID NO: 13 - DNA, 20 bp, Sus scrofa
tggtcttcatgcgcatcatg

SEQ ID NO: 14 - DNA, 52 bp, Sus scrofa
catgttcttacatgccttcaggcggctcttattcttaaataagatgtaatcc

SEQ ID NO: 15 - DNA, 53 bp, Sus scrofa
aacaaaattgattccttccttgagattggcagcttcggcaggtgagaggcaca

SEQ ID NO: 16 - DNA, 52 bp, Sus scrofa; identical to SEQ ID NO: 14

SEQ ID NO: 17 - DNA, 54 bp, Sus scrofa
aacaaaattgattccttccttgagattggcagcttcggcaggtgagaggcacaa

SEQ ID NO: 18 - DNA, 52 bp, Sus scrofa; identical to SEQ ID NO: 14

SEQ ID NO: 19 - DNA, 54 bp, Sus scrofa; identical to SEQ ID NO: 17

SEQ ID NO: 20 - DNA, 47 bp, Sus scrofa
cttatttaagaataagagccgcctgaaggcatgtaagaacatgtgca

SEQ ID NO: 21 - DNA, 102 bp, Sus scrofa
gccgaagctgccaatctcaaggaaggaatcaattttgttcgaaataagagcactggcaaggattacatcttatttaagaataagagccgccctgaaggcatg

SEQ ID NO: 22 - DNA, 101 bp, Sus scrofa
gccgaagctgccaatctcaaggaaggaatcaattttgttcgaaataagagcactggcaaggattacatcttatttaagaataagagccgcctgaaggcatg

SEQ ID NO: 23 - DNA, 102 bp, Sus scrofa; identical to SEQ ID NO: 21

SEQ ID NO: 24 - DNA, 49 bp, Sus scrofa; identical to SEQ ID NO: 10

SEQ ID NO: 25 - DNA, 54 bp, Sus scrofa; identical to SEQ ID NO: 9

SEQ ID NO: 26 - DNA, 49 bp, Sus scrofa; identical to SEQ ID NO: 10

SEQ ID NO: 27 - DNA, 55 bp, Sus scrofa
tcgtgctggatgtgggccaggatgtgctccccgatggtcttcatgcgcatcatgc

SEQ ID NO: 28 - DNA, 21 bp, Sus scrofa
ccgcctctcgtaggtgaactc

SEQ ID NO: 29 - DNA, 21 bp, Sus scrofa
tggtcttcatgcgcatcatgc

SEQ ID NO: 30 - DNA, 47 bp, Sus scrofa
tgttcttacatgccttcaggcggctcttattcttaaataagatgtaa

SEQ ID NO: 31 - DNA, 35 bp, Sus scrofa
tgagattggcagcttcggcaggtgagaggcacaac

SEQ ID NO: 32 - DNA, 46 bp, Sus scrofa
tgttcttacatgccttcaggcggctcttattcttaaataagatgta

SEQ ID NO: 33 - DNA, 36 bp, Sus scrofa
ttgagattggcagcttcggcaggtgagaggcacaac

SEQ ID NO: 34 - DNA, 23 bp, Sus scrofa
tgttcttacatgccttaagatgt

SEQ ID NO: 35 - DNA, 28 bp, Sus scrofa
cttgagattggcaggtgagaggcacaac

SEQ ID NO: 36 - DNA, 48 bp, Sus scrofa
ccgcctctcgtaggtgaactcgtcaggatgtgccttgtaccaccaggc

SEQ ID NO: 37 - DNA, 46 bp, Sus scrofa
gatgtgggccaggatgtgctccccgatggtcttcatgcgcatcatg

SEQ ID NO: 38 - DNA, 48 bp, Sus scrofa; identical to SEQ ID NO: 36

SEQ ID NO: 39 - DNA, 48 bp, Sus scrofa
ggatgtgggccaggatgtgctccccgatggtcttcatgcgcatcatgc

SEQ ID NO: 40 - DNA, 21 bp, Sus scrofa
ccgcctctcgtaggtgaactc

SEQ ID NO: 41 - DNA, 21 bp, Sus scrofa
tggtcttcatgcgcatcatgc

SEQ ID NO: 42 - DNA, 20 bp, Artificial Sequence
  Synthetic construct
ctctcgtaggtgaactcgtc

SEQ ID NO: 43 - DNA, 20 bp, Artificial Sequence
  Synthetic construct
gatgcgcatgaagaccatcg

SEQ ID NO: 44 - DNA, 23 bp, Artificial Sequence
  Synthetic construct
ccagcagtattctggggataaga

SEQ ID NO: 45 - DNA, 22 bp, Artificial Sequence
  Synthetic construct
cccagaggttacatttacccca

SEQ ID NO: 46 - DNA, 20 bp, Artificial Sequence
  Synthetic construct
taagaataagagccgcctga

SEQ ID NO: 47 - DNA, 20 bp, Artificial Sequence
  Synthetic construct
ttgagattggcagcttcggc

SEQ ID NO: 48 - DNA, 20 bp, Artificial Sequence
  Synthetic construct
gagctgccgtaaaggagctt

SEQ ID NO: 49 - DNA, 20 bp, Artificial Sequence
  Synthetic construct
ccttgatgggtaggatggcc